(19) United States
(12) Patent Application Publication
(10) Pub. No.: US 2016/0166507 A1
(43) Pub. Date: Jun. 16, 2016

(54) NANOEMULSIONS OF HYDROPHOBIC PLATINUM DERIVATIVES

(71) Applicant: NEMUCORE MEDICAL INNOVATIONS, INC., Worcester, MA (US)

(72) Inventors: Srinivas GANTA, Ellicott city, MD (US); Timothy P. COLEMAN, Wellesley, MA (US)

(73) Assignee: NEMUCORE MEDICAL INNOVATIONS, INC., Worcester, MA (US)

(21) Appl. No.: 14/907,422

(22) PCT Filed: Jul. 25, 2014
(86) PCT No.: PCT/US14/48109

§ 371 Date: Jan. 25, 2016

Related U.S. Application Data
(60) Provisional application No. 61/858,376, filed on Jul. 25, 2013.

(51) Int. Cl. A61K 9/107; A61K 47/48; A61K 49/10; A61K 49/18; A61K 31/282; A61K 31/606

(52) U.S. Cl. CPC A61K 9/1075; A61K 31/282; A61K 31/606; A61K 49/105; A61K 49/1806; A61K 47/48246; A61K 47/48215; A61K 47/48061; A61K 47/488

(57) ABSTRACT

Provided are nanoemulsion formulations useful for the delivery of hydrophobic platinum chemotherapeutic drugs to cancer patients, as well as methods of their preparation and use.

### CROSS-REFERENCE TO RELATED APPLICATION

This application claims priority to and the benefit of U.S. provisional patent application No. 61/858,376 filed on Jul. 25, 2013, the entire content of which is hereby incorporated by reference.

### STATEMENT REGARDING FEDERALLY SPONSORED RESEARCH OR DEVELOPMENT

Part of the work leading to this invention was carried out with United States Government support provided under a grant from the National Institutes of Health, Grants No. R01CA158881, U54CA151881 and R43CA144591. Therefore, the U.S. Government has certain rights in this invention.

### FIELD OF THE INVENTION

The present disclosure relates to medicine and pharmacology, and more particularly, to cancer therapy.

### BACKGROUND

Chemotherapeutic agents are widely used in cancer therapy. However, in most cases these treatments do not cure the disease. One challenge for effective therapy is that drug efficacy is often undermined by serious side-effects resulting from drug toxicities to normal tissues. Also, such treatment failures may be due to the lack of sufficient concentration and short residence time of therapeutic agents at the site of disease. (Jain (2003) Nat. Med. 9(6):685-693). Additionally, lack of target specificity contributes to systemic toxicity as the therapeutic agent builds up in non-diseased tissues.

A number of delivery systems have been developed to address these problems. For example, nanodelivery systems with site-specific binding moieties have been developed with various levels of success. Some delivery vehicles have been devised that improve drug delivery to tumors, for example Doxil (also known as Caelyx), comprising doxorubicin in polyethylene glycol (PEG)-coated liposomes. Another example is poly(epsilon-caprolactone) (PCL) nanoparticles. (Chawla et al. (2003) AAPS PharmSci. 5(1):28-34). The alkyl structure of the polymer encapsulates hydrophobic compounds. Surface modification of the colloidal carrier with an agent such as a poly(ethylene oxide)-poly(propylene oxide) (PEO-PPO-PEO) triblock copolymer can improve the solubility of the nanoparticle. However, serious side-effects of chemotherapeutic agents to normal tissue remain a challenge for effective cancer treatment.

Effective cancer therapy also suffers from the lack of early data on the delivery of a particular pharmaceutical agent to tumors and thus effectiveness. Patients often proceed with a course of treatment for an extended period of time, while suffering associated side-effects and poor quality of life, only to find out that the particular treatment is not effective.

Hydrophobicity of pharmaceutical agents limits the range of therapies for cancer treatment. Almost one third of the drugs in the United States Pharmacopeia (http://www.usp.org/) are hydrophobic and are either insoluble or poorly soluble. (Savić et al. (2006) J. Drug Target. 14(6):343-55). As a result, many potential new chemical entities are being dropped in the early phases of development because of poor solubility.

Approaches for administering hydrophobic drugs include the use of co-solvents, incorporation of complexing or solubilizing agents, chemical modification of the drug, use of micellar delivery systems such as niosomes, liposomes, and their formulation of the drug in an oily vehicle, for oral, parenteral, nasal, rectal or ophthalmic delivery. However, many of these formulations employ surfactants or co-solvents having associated toxic side-effects, and frequently, stability, sterility, and mass commercial production issues as well.

Accordingly, there exists a need for delivery systems which can efficiently deliver therapeutic levels of drug to disease sites with fewer or no side-effects. There is also a need to expand the range of therapeutics that can be used for cancer treatment. In addition, a need also exists for imaging capabilities that will allow for quick determination as to whether a patient should proceed with a particular course of treatment.

### SUMMARY

It has been discovered that formulation of certain platinum (Pt(II)) complexes into certain oil-containing nanoemulsions can increase the efficacy and efficiency of chemotherapeutic treatments. This discovery has been exploited to develop the present disclosure, which, in one aspect, is a formulation comprising an oil phase, an interfacial surface membrane, an aqueous phase, and a chemotherapeutic agent comprising a hydrophobic platinum derivative which is not cisplatin or carboplatin, and wherein the platinum chemotherapeutic agent is dispersed in the oil phase.

In some embodiments, the oil phase of the nanoemulsion formulation comprises flaxseed oil, omega-3 polyunsaturated fish oil, omega-6 polyunsaturated fish oil, safflower oil, olive oil, pine nut oil, cherry kernel oil, soybean oil, pumpkin oil, pomegranate oil, primrose oil, or any combination thereof.

In certain embodiments, the interfacial surface membrane of the nanoemulsion formulation comprises an emulsifier, a stabilizer, or a combination of thereof. In particular embodiments, the emulsifier phase of the nanoemulsion formulation comprises egg phosphatidyl choline, soy lecithin, phosphatidyl ethanolamine, phosphatidyl inositol, dimyristoylphosphatidyl choline, or dimyristoylphosphatidyl ethyl-N-dimethyl propyl ammonium hydroxide. In some embodiments, the stabilizer comprises a polyethylene glycol derivative, a phosphatide, a polyglycerol mono oleate, or any combination thereof. In certain embodiments, the polyethylene glycol (PEG) derivative comprises PEG₂₀₀₀DSPE, PEG₃₄₀₀DSPE, PEG₅₀₀₀DSPE, or any combination thereof. In particular embodiments, the polyethylene glycol in the polythylene glycol derivative has a molecular weight of from 1 kD to 20 kD, from 5 kD to 20 kD, or from 6 kD to 20 kD.

In some embodiments, the hydrophobic platinum derivative dispersed in the oil phase comprises a platinum (II) mono-fatty acid complex. In particular embodiments, the platinum (II) mono-fatty acid complex comprises platinum (II) mono-myristate (Pt-MMA), platinum (II) mono-palmitate (Pt-MPA), or platinum (II) mono-stearate (Pt-MSA), or any combination thereof. In particular embodiments, the hydrophobic platinum derivative comprises diaminocyclohexane (DACH) platinum-3,5 diiodosalicylate (Pt-SA).

In some embodiments, the nanoemulsion formulation further comprises a chemopotentiator. In particular embodiments, the chemopotentiator comprises ceramide (CER) or a derivative thereof. In certain embodiments, the chemopotentiator comprises C6-ceramamide. In other embodiments, the chemopentiator comprises C6-ceramamide and the platinum derivative comprises Pt-MMA. In certain embodiments, C6-ceramde and Pt-MMA have a combination index of from 0.1 to 0.9 or about 0.3326. In some embodiments, the chemopentiator comprises C6-ceramamide and the platinum derivative comprises Pt-MPA. In certain embodiments, C6-ceramide and Pt-MPA have a combination index of from 0.1 to 0.9 or about 0.5746.

In some embodiments, the nanoemulsion formulation further comprises a targeting ligand. In certain embodiments, the targeting ligand comprises an EGFR-targeting ligand, a folate receptor-targeting ligand, or a combination thereof. In particular embodiments the targeting ligand comprises the amino acid peptide Y-H-W-Y-G-Y-T-P-Q-N-V-I (SEQ ID NO:1) (peptide 4), an anti-EGFR immunoglobulin, or EGFR binding fragment thereof, EGa1-PEG, or any combination thereof. In certain embodiments, the PEG in the targeting ligand EGa1-PEG has a molecular weight of from 1 kD to 20 kD, from 5 kD to 20 kD, or from 10 kD to 20 kD. In other embodiments, the folate receptor-targeting ligand comprises DSPE-PEG-cysteine-folic acid, DSPE-PEG(2000) folate, DSPE-PEG(5000) folate, an anti-folate receptor immunoglobulin or folate receptor-binding fragment thereof, or any combination thereof.

In some embodiments, the nanoemulsion formulation further comprises an imaging agent. In particular embodiments, the imaging agent is an MRI contrasting moiety. In certain embodiments the MRI contrasting moiety comprises gadnolinium, iron oxide, iron platinum or manganese or any combination thereof. In certain embodiments, the MRI contrasting moiety comprises Gd-DTPA-PE, Gd-DOTA-PE, Gd-PAP-DOTA, or any combination thereof.

In another aspect, the present disclosure provides a method of imaging a cancer in a patient. The method comprises administering to the patient an amount of the nanoemulsion formulation sufficient to image the tumor.

In yet another aspect, the present disclosure provides a method of inhibiting the growth of, or killing, a cancer cell, comprising contacting the cancer cell with an amount of a nanoemulsion formulation according to the specification that is toxic to, and/or which inhibits the growth of, or kills, the cancer cell. In some embodiments, the cancer cell is in a mammal, and the nanoemulsion is administered to the mammal in a therapeutically effective amount.

### DESCRIPTION OF THE FIGURES

The foregoing and other objects of the present disclosure, the various features thereof, as well as the invention itself may be more fully understood from the following description, when read together with the accompanying drawings in which:

FIG. 1 is a schematic representation of a generic nanoemulsion formulation of the present disclosure;

FIG. 2 is a schematic representation of a non-limiting mode for preparing a platinum (II) complex which can be used in a nanoemulsion formulation;

FIG. 3 is a representation of a RAMAN spectrum of cisplatin-monomyristate (Pt-MMA);

FIG. 4 is a representation of a RAMAN spectrum of cisplatin-monopalmitate (Pt-MPA);

FIG. 5 is a representation of an NMR spectrum of cisplatin-monomyristate (Pt-MMA);

FIG. 6 is a representation of an NMR spectrum of cisplatin-palmitate (Pt-MPA);

FIG. 7 is a representation of an NMR spectrum of cisplatin-monostearate (Pt-MSA);

FIG. 8 is a representation of an NMR spectrum of cisplatin-monosalicylate (Pt-SA);

FIG. 9 is a diagrammatic representation of one non-limiting method of preparing an EGFR targeted nanoemulsion formulation;

FIG. 10A is a graphic representation of a DLS plot of an EGFR-targeted nanoemulsion showing the particle size;

FIG. 10B is a representation of a transmission electron micrograph (TEM) of an EGFR-targeted nanoemulsion formulation showing the particle size;

FIG. 11 is a schematic representation of a representative non-limiting method of preparing DACH-Pt-diiodo salicylic acid;

FIG. 12 is a schematic representation of one non-limiting method of synthesizing EGFR-MAL-PEG-DSPE;

FIG. 13 is a schematic representation of one non-limiting mode of synthesizing DSPE-PEG-Cys-FA;

FIG. 14 is a schematic representation of one non-limiting method of preparing Gd⁺³-DTPA-PE;

FIG. 15A is a representation of the fluorescent images of SKOV3 cells 5 minutes after treatment with a fluorescently labeled, non-targeted nanoemulsion formulation.

FIG. 15B is a representation of the fluorescent images of SKOV3 cells 15 minutes after treatment with a fluorescently labeled, non-targeted nanoemulsion formulation.

FIG. 15C is a representation of the fluorescent images of SKOV3 cells 30 minutes after treatment with a fluorescently labeled, non-targeted nanoemulsion formulation.

FIG. 15D is a representation of the fluorescent images of SKOV3 cells 5 minutes after treatment with a fluorescently labeled, EGFR-targeted nanoemulsion formulation.

FIG. 15E is a representation of the fluorescent images of SKOV3 cells 15 minutes after treatment with a fluorescently labeled, EGFR-targeted nanoemulsion formulation.

FIG. 15F is a representation of the fluorescent images of SKOV3 cells 30 minutes after treatment with a fluorescently labeled, EGFR-targeted nanoemulsion formulation.

FIG. 16 is a graphic representation of the therapeutic efficacy of an EGFR-targeted nanoemulsion formulation according to the disclosure, relative to control treatments (none or cisplatin as measured by the survival rate of treated animals in days post-treatment;

FIG. 17 is a graphic representation of the concentration of Pt in plasma over time after treatment with cisplatin, an EGFR-targeted Pt-MMA nanoemulsion formulation, a non-targeted Pt-MMA nanoemulsion formulation, a Gd-labeled, EGFR-targeted Pt-MMA, or a Gd-labeled, non-targeted Pt-MMA nanoemulsion formulation administered intravenously to CD-1 mice; and

FIG. 18 is a series of representations of magnetic resonance images (MRI) of mice treated with Gd3+-DTPA-PE, Magnevist™ (Mag), Gd3+-DTPA-PE, non-targeted nano-3+ emulsion formulation (NT), or Gd-DTPA-PE, EGFR-targeted nanoemulsion formulation (T).

### DESCRIPTION

Throughout this application, various patents, patent applications, and publications are referenced. The disclosures of these patents, patent applications, and publications in their entireties are hereby incorporated by reference into this application in order to more fully describe the state of the art as known to those skilled therein as of the date of the invention described and claimed herein. The instant disclosure will govern in the instance that there is any inconsistency between the patents, patent applications, and publications and this disclosure.

### DEFINITIONS

For convenience, certain terms employed in the specification, examples, and appended claims are collected here. Unless defined otherwise, all technical and scientific terms used herein have the same meaning as commonly understood by one of ordinary skill in the art to which this invention belongs. The initial definition provided for a group or term herein applies to that group or term throughout the present specification individually or as part of another group, unless otherwise indicated.

The articles “a” and “an” are used herein to refer to one or to more than one (i.e., to at least one) of the grammatical object of the article. By way of example, “an element” means one element or more than one element.

The term “or” is used herein to mean, and is used interchangeably with, the term “and/or,” unless context clearly indicates otherwise.

The term “about” is used herein to mean approximately, in the region of, roughly, or around. When the term “about” is used in conjunction with a numerical range, it modifies that range by extending the boundaries above and below the numerical values set forth. In general, the term “about” or “approximately” is used herein to modify a numerical value above and below the stated value by a variance of 20%.

The expression “at least one” is used herein to mean one or more and thus includes individual components as well as mixtures/combinations.

“Anticancer agent” or “chemotherapeutic agent” is an agent that prevents or inhibits the development, growth or proliferation of malignant cells.

“Cancer” is the uncontrolled growth of abnormal cells.

“Stable Pt-containing formulation” is a formulation containing a Pt-containing compound or ion wherein the compound or ion is stable for transformation for a time sufficient to be therapeutically useful.

“Stabilizer” is an agent that prevents or slows the transformation or deactivation of a Pt-containing compound or ion in a Pt-containing formulation.

“Patient” is a human or animal in need of treatment for cancer.

“DACH” Cylohexane-1,2-diammine

“Capryl” Caprylic acid residue (OCOC7H15)

“Cap” Capric acid residue (OCOC9H19)

“Lau” Lauric acid residue (OCOC11H23)

“Myr” Myristic acid residue (OCOC13H27)

“Pal” Palmitic acid residue (OCOC15H31)

“Ste” Stearic acid residue (OCOC17H35)

“Stol” Myristoleic acid residue (OCOC13H25)

“Tol” Palmitoleic acid residue (OCOC15H29)

“Sap” Sapienic acid residue (OCOC15H29)

“Oleic” Oleic acid residue (OCOC17H33)

“Ela” Elaidic acid residue (OCOC17H33)

“Vac” Vaccenic acid residue (OCOC17H33)

“CDDP” Cis-dichlorodiammine Pt (II)

“DACHP” Dichloro cyclohexane-1,2-diammine Pt (II)

An “organic substituent” is defined as a carbon atom or a carbon containing molecule substituted for a hydrogen.

A “bivalent organic group” is defined as a carbon atom or carbon containing molecule capable of forming two bonds with other atoms or molecules.

A “coordinate bond”, also known as a dipolar or dative covalent bond is a kind of 2-center, 2-electron covalent bond in which the two electrons are from the same atom.

A “combination index” is defined as an isobologram equation to study combination drug effects on cells and to determine whether the drug combination produced enhanced efficacy in the form of an additive, synergistic, or antagonistic effect on cells.

“Pt compound” or “Pt (II) complex” as used herein encompasses Pt derivatives, including salts having anticancer Pt activity. One non-limiting Pt compound is cisplatin.

“Stabilizer” as used herein means an agent that prevents or slows the transformation or deactivation of a Pt-containing compound or ion in a Pt-containing nanoemulsion formulation.

“Patient” as used herein means a human or animal in need of treatment for cancer.

“Nanoemulsion formulation” as used herein means a novel nanoemulsion (NE) comprising an oil phase; an interfacial surface membrane; an aqueous phase; and a Pt compound dispersed in the oil phase.

“Nanoemulsion” as used herein means a colloidal dispersion comprised of omega-3, -6 or -9 fatty acid rich oils in an aqueous phase and thermo-dynamically stabilized by amphiphilic surfactants, which make up the interfacial surface membrane, produced using a high shear microfluidization process usually with droplet diameter within the range of about 80-220 nm.

“Oil phase” or “lipid phase” as used herein means the internal hydrophobic core of the nanoemulsion in which a Pt-compound is dispersed and refers either to a single pure oil or a mixture of different oils present in the core. The oil phase is comprised of generally regarded as safe grade, parenterally injectable excipients generally selected from omega-3, omega-6 or omega-9 polyunsaturated unsaturated fatty acid (PUFA) or monounsaturated fatty acid rich oils.

“Aqueous phase” is comprised of isotonicity modifiers and pH adjusting agents in sterile water for injection and forms as an external phase of the nanoemulsion formulation in which the oil phase is dispersed.

“Amphiphilic molecule or amphiphilic compound” as used herein means any molecule of bipolar structure comprising at least one hydrophobic portion and at least one hydrophilic portion. The hydrophobic portion distributes into the oil phase and hydrophilic portion distributes into aqueous phase forming an interfacial surface membrane and has the property of reducing the surface tension of water (g<55 mN/m) and of reducing the interface tension between water and an oil phase. The synonyms of amphiphilic molecule are, for example, surfactant, surface-active agent and emulsifier.

“Amphiphilic or amphiphile” as used herein means a molecule with both a polar, hydrophilic portion and a non-polar, hydrophobic portion.

“Primary emulsifiers” as used herein means amphiphilic surfactants that constitute a major percentage of amphiphilic surfactants of the nanoemulsion formulation wherein they stabilize the formulation by forming an interfacial surface membrane around oil droplets dispersed in water, and further allow for surface modification with targeting ligands and imaging agents.

“Co-emulsifiers” as used herein means amphiphilic surfactants used in conjunction with primary emulsifiers where they associate with the interfacial surface membrane, effectively lowering the interfacial tension between oil and water, and help in the formation of stable nanoemulsion formulations.

“Stabilizers” or “stealth agents” as used herein mean lipidated polyethylene glycols (PEG) where the lipid tail group distributes into the oil phase and hydrophilic PEG chains distribute into the aqueous phase of a nanoemulsion formulation, providing steric hindrance to mononuclear phagocytic system (MPS) cell uptake during the blood circulation, thus providing longer residence time in the blood and allowing for enhanced accumulation at tumor site through leaky tumor vasculature, a phenomenon termed as enhanced permeability and retention effect, largely present in wide variety of solid tumors. Other representative examples are a phosphatide, and a polyglycerol mono oleate,

“Targeting agents” as used herein are molecules which direct a nanoemulsion particle toward a tumor cell or in the vicinity thereof. Such targeting agents allow for interaction with tumor cells in vivo, forming a ligand-receptor complex, which is taken up by the tumor cells.

“Imaging agents” as used herein encompass metal ions (eg. gadolinium, iron and manganese), which provide contrast to visualize a disease site using magnetic resonance imaging (MRI), and the near infra-red fluorescent dyes (eg. DiR), which provide fluorescence at the disease site. These agents are either linked to lipidated chelates (eg. DTPA-PE and DOTA-PE) and incorporated in the nanoemulsion formulation at its interfacial surface membrane or are loaded inside the oil core of the nanoemulsion formulation according to the disclosure.

“Isotonicity modifiers” as used herein means agents that provide an osmolality (285-310 mOsm/kg) to the nanoemulsion formulation, thus maintaining isotonicity for parenteral injection.

“pH modifiers” as used herein means buffering agents that adjust the pH of nanoemulsion formulation to a value of about pH 6-7.4, thus preventing the hydrolysis of phospholipids upon storage.

“Preservatives” as used herein means antimicrobial agents that when added to the nanoemulsion formulation at about 0.001-005% w/v prevent bacterial growth during the storage of nanoemulsion formulation.

“Antioxidants” as used herein means agents that stop oxidation of oils comprised of fatty acids, thus preventing rancidification of oil phase and destabilization of the nanoemulsion formulation.

“Chemopotentiator” as used herein means a drug or chemotherapeutic agent used in combination with other drugs or chemotherapeutic agents to enhance, increase or strengthen the effect, for instance decrease the IC₅₀, of the drug or chemotherapeutic agent.

### 1. Nanoemulsion Formulations

The present disclosure provides novel nanoemulsion formulations useful for cancer treatment and imaging of tumors and cancer cells. This formulation comprises an oil phase, an interfacial surface membrane, an aqueous phase, and a chemotherapeutic agent comprising a hydrophobic platinum derivative, which is not cisplatin or carboplatin, wherein the platinum chemotherapeutic agent is dispersed in the oil phase.

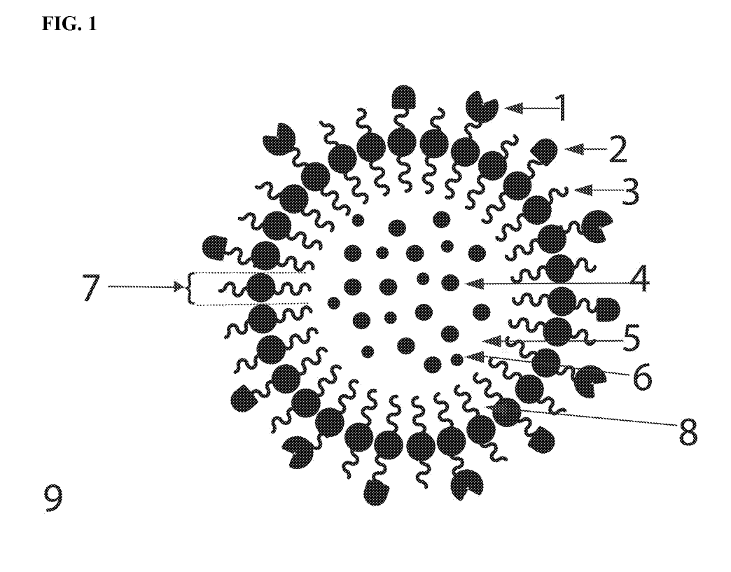

FIG. 1 is a non-limiting schematic representation of a nanoemulsion formulation of the present disclosure. In this figure, 4 represents a chemotherapeutic agent comprising hydrophobic platinum derivatives dispersed in the oil phase 5 of the nanoemulsion formulation. 5 is encapsulated within the interfacial membrane 7 which comprise emulsifiers 8 and stabilizers 3. The polar, hydrophilic portions of the amphiphiles of the interfacial surface membrane project into the aqueous phase 9, and the non-polar, hydrophobic portions of these amphiphiles project into the oil phase 5. 1 represents a targeting ligand linked to stabilizers 3 in the interfacial surface membrane, 2 represents an imaging agent attached to an emulsifier 8 in the interfacial surface membrane 7 and 6 represents a chemopotentiator dispersed in the oil phase 5 of the nanoemulsion formulation.

The nanoemulsion formulation of the present disclosure may also comprise a co-emulsifier, a preservative, an antioxidant, a pH adjusting agent, an isotonicity modifier, or any combination thereof).

Various non-limiting examples of the components of the nanoemulsion formulation of the present disclosure and their corresponding proportions are provided in Table I and discussed below.

TABLE I
Component Proportions for Representative Formulations

| Category | Examples | % w/w |
|---|---|---|
| Lipid phase | flaxseed oil (omega-3 and omega-6 polyunsaturated components), | 5-30 |
| | fish oil (omega-3 and omega-6 polyunsaturated components), | |
| | safflower oil, | |
| | olive oil, | |
| | pine nut oil, | |
| | cherry kernel oil, | |
| | soybean oil (omega-6 polyunsaturated component), | |
| | pomegranate oil, | |
| | pumpkin oil, | |
| | primrose oil, | |
| | diacetyl mono glycerides, | |
| | medium and long chain triglycerides | |
| Primary emulsifiers | egg lecithin (egg phosphatidyl choline, ePC), | 0.5-5.0 |
| | soy lecithin (sPC), | |
| | phosphatidyl ethanolamine, | |
| | phosphatidyl inositol, | |
| | dimyristoylphosphatidyl choline, | |
| | dimyristoylphosphatidyl ethyl-N-dimethyl propyl ammonium | |
| | hydroxide | |
| Co-emulsifiers | nonionic surfactants—tween 80, span 80, span 65, myrj 52 | 0.1-1.0 |
| | block copolymer—poloxamer 188, poloxamer 407, Acetylated- | |
| | mono-glycerides, | |
| | poly(oxyethylene)-6-glycerol trioleate, | |
| | poly(oxyethylene)-6-glycerol linolase | |
| Stealth Agents | polyethylene glycols derivatives—PEG₂₀₀₀DSPE, PEG₅₀₀₀DSPE | 0.1-0.75 |
| | phosphatides, polyglycerol mono oleate, | |

A. Chemotherapeutic Agents

The novel pharmaceutical formulations according to the disclosure contain certain platinum (II) complexes. One representative platinum (II) complex is, e.g., a liposoluble platinum (II) mono-fatty acid complex, having improved lipophilicity and stability and thus effective for therapeutic administration in the nanoemulsion formulations of the present disclosure. Other useful agents include hydrophobic prodrugs containing platinum, Pt polymer conjugates, and systems such as encapsulated Pt (II) complex nanocarrier formulations having targeting ligands on their surface and that can be designed for controlled release. The present disclosure excludes the platinum derivatives cisplatin and carboplatin.

In general, the Pt (II) complex has the following general formula (I):

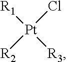

wherein

R₁ and R₂ are each an ammine (NH₃) which optionally has an organic substituent A (A-NH₂):

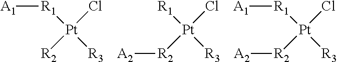

R₁ and R₂ may be identical or different, are linked to platinum via coordinate bonds, and optionally may be linked together via a bivalent organic group B (NH₂—B—NH₂):

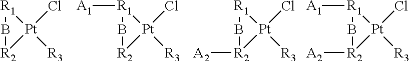

The substituent R₃ in the general formula (I) may be a saturated or unsaturated higher fatty acid having 8 to 24 carbon atoms. Non-limiting examples thereof are saturated fatty acids such as caprylic acid, capric acid, lauric acid, myristic acid, palmitic acid, and stearic acid, and unsaturated higher fatty acids having 8 to 24 carbon atoms, such as myristoleic acid, palmitoleic acid, sapienic acid, oleic acid, elaidic acid, and vaccenic acid. The substituent for R₃ and Cl in the general formula (I) may be 3,5-diiodosalicylic acid.

Accordingly, possible non-limiting structural variations of the complex include a complex with no organic substituents and a bivalent organic group that may or may not be present; a complex with an organic substituent attached to R₁ and a bivalent organic group that may or may not be present; a complex with an organic substituent attached to R₂ and a bivalent organic group that may or may not be present; and a complex with an organic substituent attached to both R₁ and R₂ and a bivalent organic group that may or may not be present.

Non-limiting examples of the complex include ones in which the organic substituent (A) is a member selected from the group comprising alkyl groups having 1 to 5 carbon atoms, such as an isopropyl group, and cycloalkyl groups having 3 to 7 carbon atoms, such as a cyclohexyl group.

Non-limiting examples of the complex include ones in which the bivalent organic group (B) is a member selected from the group comprising cycloalkylene groups; alkylene groups having 2 or 3 carbon atoms, eventually substituted with an alkyl group having 1 to 5 carbon atoms, an alkylene group having 2 to 6 carbon atoms, or phenyl group; and a 1, 2-phenylene group eventually substituted with an alkyl or alkoxyl having 1 to 5 carbon atoms or a halogen atom. Other non-limiting examples of the bivalent organic group include such groups as 1,2-cyclohexylene, 2,2-pentamethylene-trimethylene:

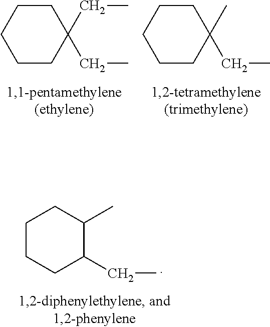

In the liposoluble Pt (II) complex, isomers, i.e., cis- and trans-form, are present when the bivalent organic group is 1,2-cyclohexylene or other such similar groups. In this respect, the complex may be in the form of cis or trans or the mixture thereof.

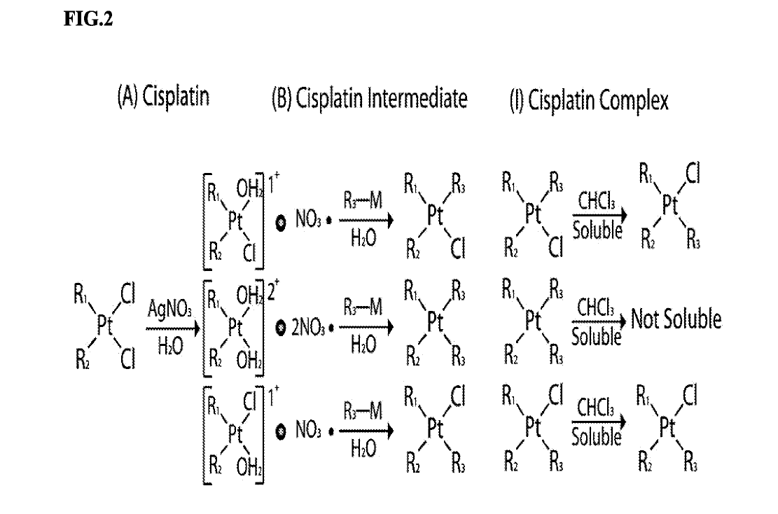

A non-limiting example of the preparation of a liposoluble Pt (II) complex of the present disclosure, represented by the general formula (I), is shown in FIG. 2 and may be prepared using various intermediates and component constituents, for example, as described in Examples 1-4 below. According to this aqueous reaction method, a cis-dichloro-diammine Pt (II) complex (A) (Connors et al. (1972) Chem. Biol. Interact. 5:415) is first converted to monohydrate or dihydrate complex intermediate by treatment with a 1:1 molar ratio of a suitable reagent and then the resulting intermediate (B) monohydrate or dihydrate is subjected to the reaction with a 1:1 molar ratio of a desired alkali metal salt of saturated or unsaturated higher fatty acid to form a saturated or unsaturated higher fatty acid derivative (I) of diammine platinum (II).. This reaction results in the formation of both mono and di-fatty acid derivatives. Cisplatin mono-fatty acids are then separated out by dissolving the mixture in chloroform, in which mono-fatty acids are soluble but di-fatty acid derivatives are not soluble. The first step of the reaction may be carried out using any suitable reagent such as silver nitrate (AgNO₃) that will form a halide that is insoluble in the reaction medium and the alkali metal used for the salt of the saturated or unsaturated higher fatty acid may be, but is not limited to, sodium or potassium.

The reaction, in which the complex (A) is converted to the monohydrate or dihydrate intermediate (B) of the platinum (II) complex, is performed under light-shielding conditions, and the reaction occurs in approximately 3 weeks at RT. To facilitate dissolution in a reaction medium, complex (A) is heated to approximately 70° C. prior to the addition of the second reagent.

The reaction (B)-(I) is also performed under light-shielding conditions, and takes approximately 3 weeks at RT to complete.

The Pt (II) complex thus obtained is liposoluble, and thus is available for use as an anticancer agent having a high specificity and selectivity to cancer cells. Moreover, its liposolubility makes it useful as a slowly and steadily released and sustained medicine. The complex may be combined with a carrier such as an emulsion, nanoemulsion or liposome to target it more specifically to a cancer in vivo. The Pt (II) complexes are effective against a wide range of known cancers including breast, colorectal, lung, ovarian, gastric, renal, and prostate cancer. It has been discovered that the encapsulation of hydrophobic Pt (II) complexes in the nanoemulsion formulations of the present disclosure aid in mitigating undesirable side-effects known to sometimes accompany their use.

Tables II and III show the solubility of di-fatty acid platinum complexes relative to a monofatty acid platinum complex.

TABLE II
Solubility of Di-Fatty Acid Complex

| Compound | Water | CHCl₃ | DMC | THF | DMSO | MeOH | EtOH | ACN | DMF |
|---|---|---|---|---|---|---|---|---|---|
| Cisplatin- | − | ++ | − | − | − | + | − | − | + |
| dimyristate | | | | | | | | | |
| Cisplatin- | − | + | + | − | − | − | − | − | − |
| dipalmitate | | | | | | | | | |
| Cisplatin- | − | − | − | − | − | − | − | − | − |
| distearate | | | | | | | | | |
| Cisplatin- | − | − | − | − | − | − | − | − | |
| dioleate | | | | | | | | | |
| Cisplatin- | − | − | − | − | − | − | − | − | |
| dioctanoate | | | | | | | | | |
| Cisplatin- | − | − | − | − | − | − | − | − | |
| dilinoleate | | | | | | | | | |

As shown, the liposolubility of the Pt (II) complex of the present disclosure is improved compared to that of the Pt (II) di-fatty acid complex because it has significantly improved lipophilicity with regard to certain compounds. Thus, it allows for Pt encapsulation in certain formulations of cancer treating lipid emulsions, nanoemulsions, liposomes, or other suitable stable nanocarriers for which Pt encapsulation was previously unavailable. The solubility of the Pt (II) di-fatty acid complex can be seen in Table II; while the solubility of certain embodiments of the Pt (II) complex of the present disclosure can be seen in Table III (wherein “+” indicates soluble and “−” indicates insoluble).

The cisplatin di-fatty acid derivatives were found to have poor lipophilicity as shown in Table II. Formulations made with these compounds underwent destabilization during storage, as evidenced by phase separation and drug sediment formation. The poor lipophilicity of these compounds is due to the presence of a di-fatty acid structure, which causes them to be too hydrophobic.

TABLE III
Solubility of Mono-Fatty Acid Complex

| Compound | Water | CHCl₃ | DMC |
|---|---|---|---|
| Cisplatin-mono- | − | ++++ | + |
| myristate | | | |
| Cisplatin-mono- | − | ++++ | + |
| palmitate | | | |
| Cisplatin-mono- | − | ++++ | + |
| stearate | | | |

In contrast, as seen in Table III, Pt derivatives with the mono-fatty acid structure of the present disclosure gave good lipophilicity, thus making them suitable for administration in additional types of lipid emulsions, nanoemulsions, liposome or other suitable stable nanocarriers.

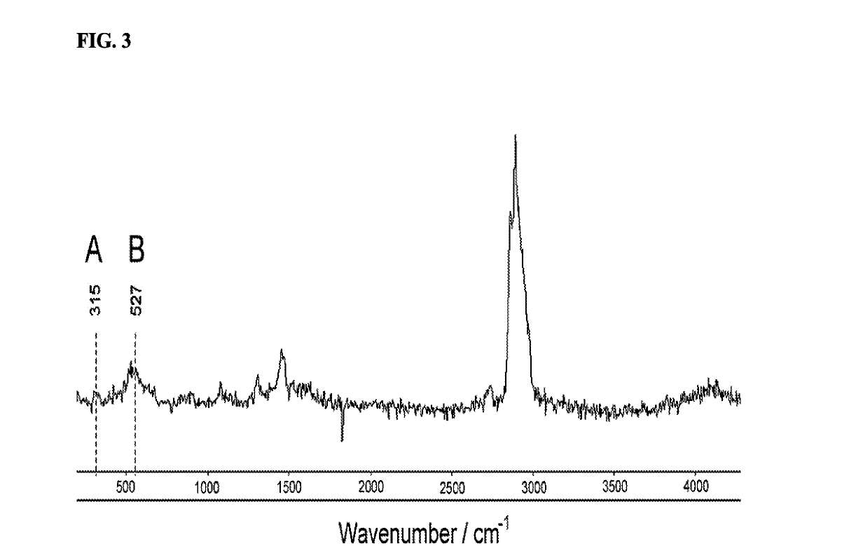

FIGS. 3-4 and FIGS. 5-8 display the RAMAN spectra and the NMR spectra, respectively, for particular embodiments of the Pt (II) complex of the present disclosure having the following general formula (I):

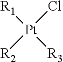

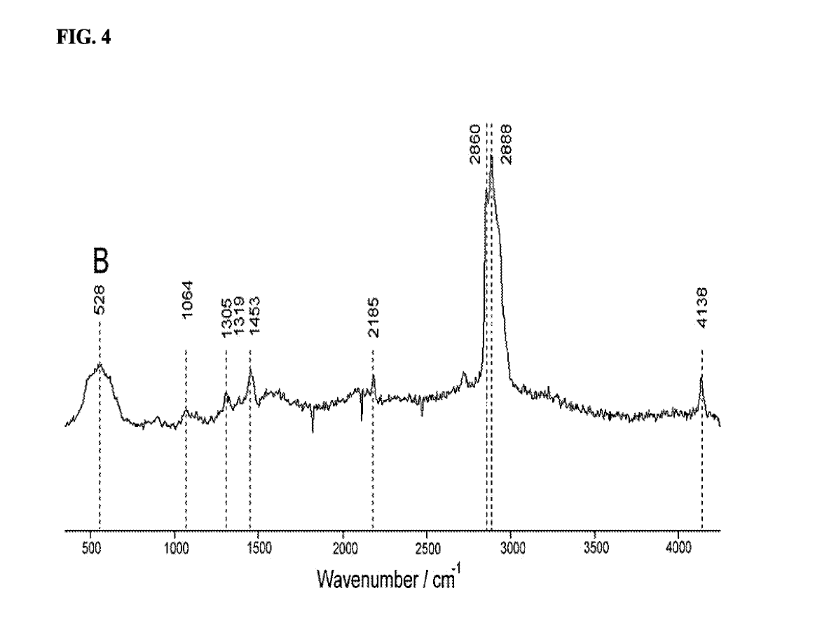

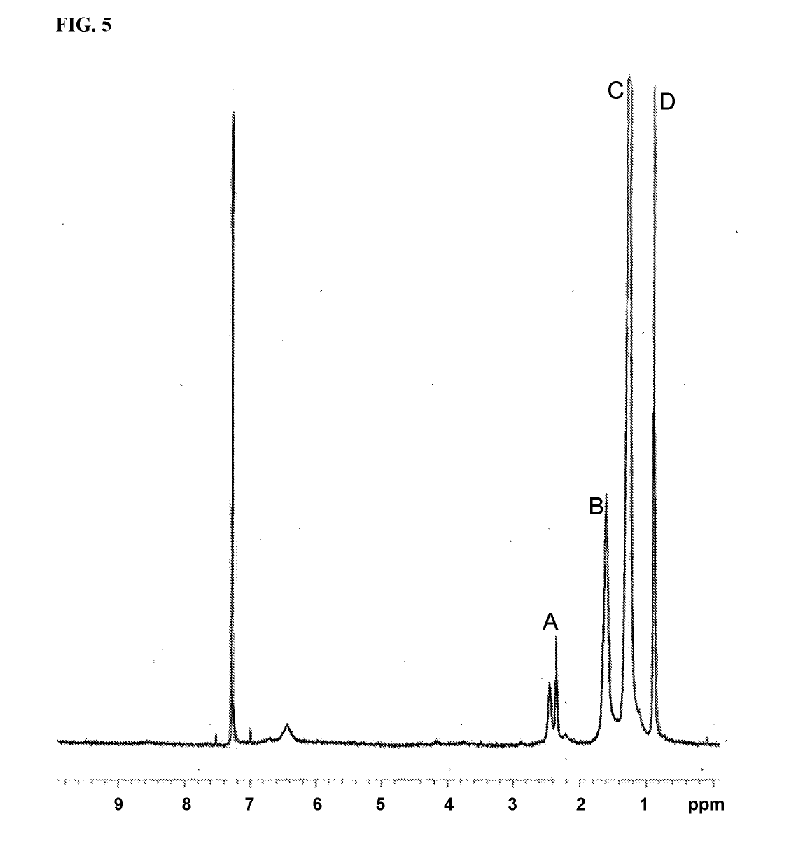

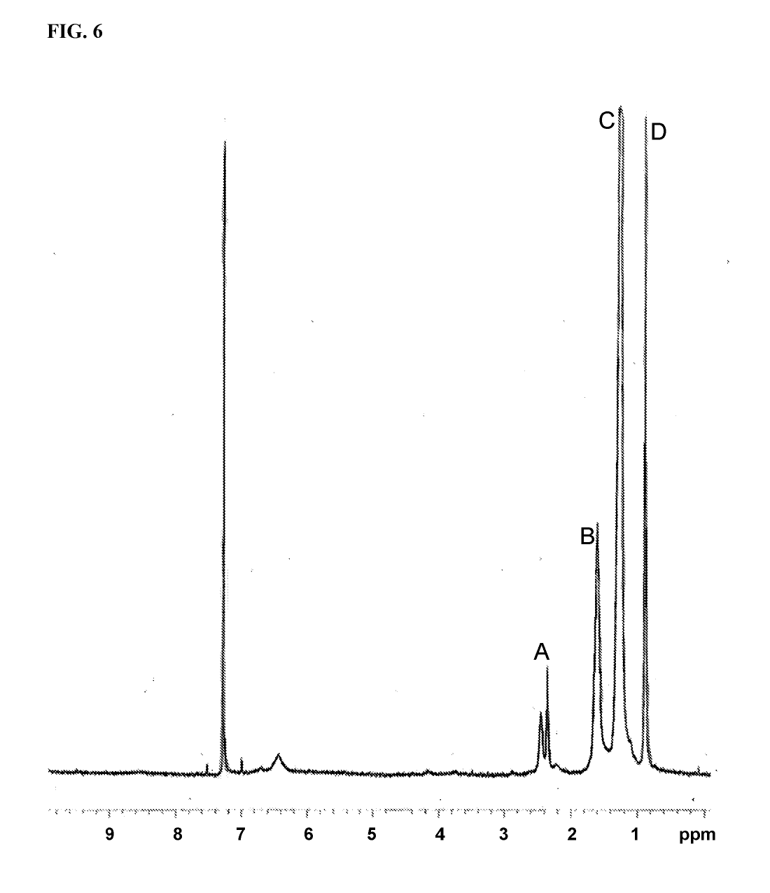

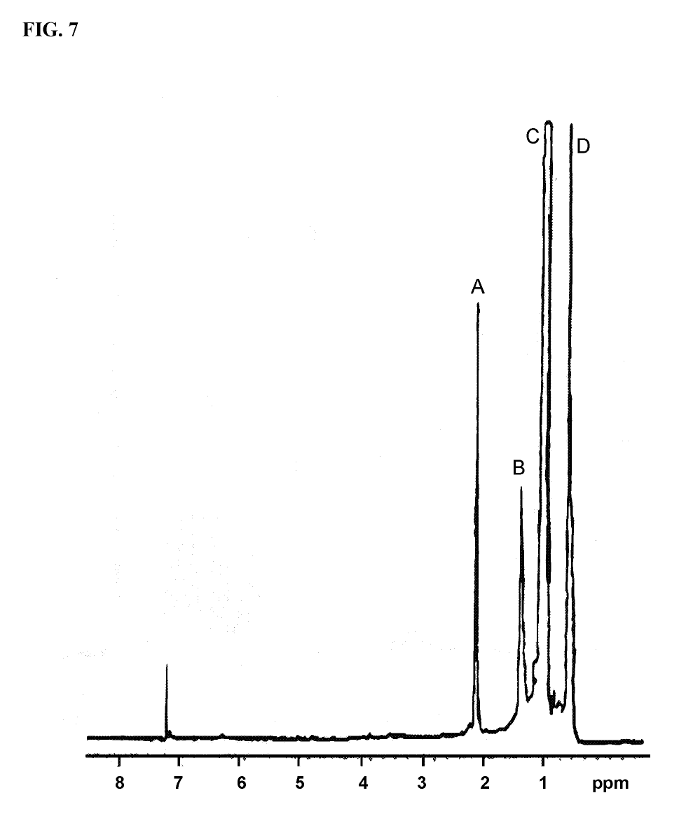

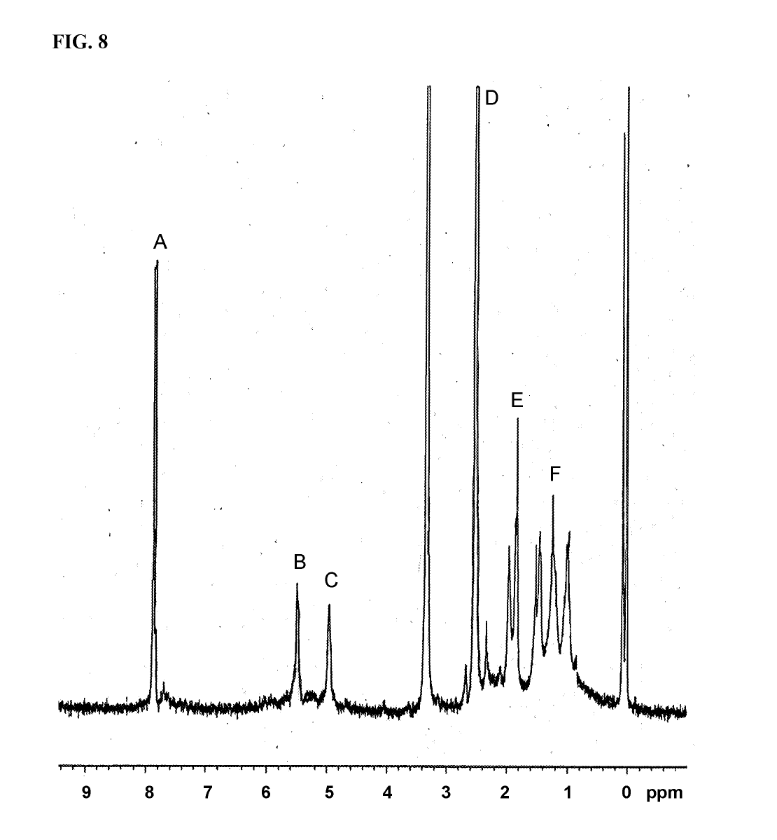

In these particular embodiments, a central Pt atom is bonded to one ammonia group (NH₃) in the R₁ position, one ammonia group (NH₃) in the R₂ position, one chlorine atom (Cl) as shown, and one myristic fatty acid chain (CH₃(CH₂)₁₂COOH), one palmitic fatty acid chain (CH₃(CH₂)₁₄COOH), or one stearic fatty acid chain (CH₃(CH₂)₁₆COOH) in the R₃ position or one salicylic acid C₆H₄(OH)(I₂)COOH replacing the Cl and R₃, thus creating a Pt (II) mono-fatty acid or mono-salicylic acid complex. The RAMAN spectra shown in FIGS. 3-4 and confirm the presence of the one Pt-chlorine bond and the two Pt-nitrogen bonds. Peak A of the RAMAN spectrum corresponds to the Pt-chlorine bond and peak B corresponds to the two Pt-nitrogen bonds. The NMR spectra in FIGS. 5-8 confirm the structure of a myristic fatty acid chain, palmitic fatty acid chain, stearic fatty acid chain and a salicylic acid moiety, respectively in the R₃ position. In FIG. 5, Peak A of the NMR spectrum corresponds to a CH₂ group, peak B also corresponds to a CH₂ group, peak C corresponds to a (CH₂)₁₀ group, and peak D corresponds to a CH₃ group of the myristic acid chain of Pt-MMA. In FIG. 6, Peak A of the NMR spectrum corresponds to a CH₂ group, peak B also corresponds to a CH₂ group, peak C corresponds to a (CH₂)₁₀ group, and peak D corresponds to a CH₃ group of the palmitic acid chain of Pt-MPA. In FIG. 7, peak A of the NMR spectrum corresponds to a CH₂ group, peak B also corresponds to a CH₂ group, peak C corresponds to a (CH₂)₁₀ group, and peak D corresponds to a CH₃ group of the steric acid chain of Pt-MSA. In FIG. 8, peak A of the NMR spectrum corresponds to the protons of the aromatic ring of 3,5-diiodosalicylate, peaks B and C correspond to the CH protons, and peaks D, E and F corresponds to a CH₂ protons of the cyclohexyl ring of Pt-SA.

The nanoemulsion formulations of the present disclosure may comprise a chemopotentiator, further comprising ceramide (CER) or CER derivatives, which enhance apoptosis (programmed cell death) and the ability of chemotherapeutic agents to kill cancer cells. The structure of C-6 CER is as follows:

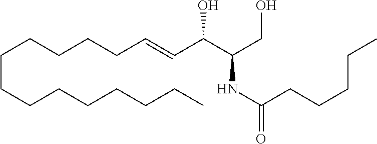 [SMILES: CCCCCCCCCCCCC/C=C/[C@H](O)[C@@H](CO)NC(=O)CCCCC]

The regulation of CER levels involves many enzymes including CER synthase, glucosylceramide synthase (GCS), beta-glucosidase, ceramidase, sphingomyelin synthase, and sphingomyelinase, all of which are responsible for the generation of CER, glucosylceramide, sphingosine, and sphingomyelin. GCS is one enzyme that provides a major route for CER clearance. As an enzyme that catalyzes the first step in glycosphingolipid synthesis, GCS transfers UDP-glucose to CER to form glucosylceramide.

Over-expression of GCS is associated with decreased rates of apoptosis in many cancer types. Endogenous addition of CER can significantly enhance the apoptotic potential of chemotherapies, thus improving efficacy. (Shabbits et al. (2003) Biochim. Biophys. Acta, 1612:98-106). Replacement of CER mediates induction of apoptosis via the inhibition of Akt pro-survival pathways, mitochondrial dysfunction, and stimulation of caspase activity, ultimately leading to DNA fragmentation.

While CER's chemopotentiator benefits seem to enhance the efficacy of chemotherapeutic agents, there are obstacles to the delivery of CER. First, CER's effectiveness is limited due to its hydrophobicity and possible precipitation when administered in aqueous solutions. In addition, the existence of a second aliphatic chain hinders cellular permeability. Also, free CER is susceptible to metabolic inactivation by specific enzymes in the systemic circulation. Accordingly, measures, which avoid these obstacles are useful. In this regard, the present nanoemulsion formulations exploit the chemopotentiator benefits of CER by providing increased solubility, intracellular permeability, and protection from systemic enzymatic degradation.

B. Oil Phase

One component of the nanoemulsion formulation according to the present disclosure is an oil or lipid phase comprising individual oil droplets, which represents the internal hydrophobic or oil core. The oil phase may be a single entity or a mixture. The average size of the oil droplets in the oil phase ranges from about 5 nm to 500 nm.

A wide variety of oils and methods for forming nanoemulsion formulations therefrom are known in the art of drug delivery. The oil phase of the disclosed nanoemulsion formulations may include at least one PUFA-rich oil, for example, a first oil that may contain polyunsaturated oil, for example linolenic acid, and optionally an oil that may be for example a saturated fatty acid, for example icosanaic acid.

Any oil can be used in accordance with the present invention. Oils can be natural or unnatural (synthetic) oils. Oils can be homogeneous or oils comprising two or more monounsaturated fatty acid or PUFA-rich oils. Contemplated oils may be biocompatible and/or biodegradable.

Biocompatible oils do not typically induce an adverse response (such as, but not limited to, an immune response with significant inflammation and/or acute rejection) when inserted or injected into a living subject. Accordingly, the therapeutic nanoemulsion formulations contemplated herein can be non-immunogenic.

Biocompatibility typically refers to the lack of acute rejection of a material by at least a portion of the immune system. A non-biocompatible material implanted into a subject provokes an immune response that can be severe enough to cause rejection of the material by the immune system that cannot be adequately controlled, and often is of a degree such that the material must be removed from the subject.

One simple test to determine biocompatibility is to expose a nanoemulsion formulation to cells in vitro. Biocompatible oils in the nanoemulsion formulation typically will not result in significant cell death at moderate concentrations, e.g., 50 μg/10⁶ cells. For example, these biocompatible oils can cause less than about 20% cell death when exposed to or taken up by, fibroblasts or epithelial cells. Non-limiting examples of biocompatible oil useful in nanoemulsion formulations of the present disclosure include alpha linolenic acid, pinolenic acid, gamma linolenic, linoleic acid, oleic acid, icosenoic acid, palmitic acid, stearic acid, icosanaic acid, and derivatives thereof.

The biocompatible oils may be biodegradable, i.e., able to degrade, chemically and/or biologically, within a physiological environment, such as within the body. As used herein, “biodegradable” oils are those that, when introduced into cells, are broken down by the cellular machinery (biologically degradable) and/or by a chemical process, such as hydrolysis, (chemically degradable) into components that the cells can either reuse or dispose of without significant toxic effect on the cells. Both the biodegradable oils and their degradation byproducts can be biocompatible.

A useful oil is flax seed oil which is a biocompatible and biodegradable oil of alpha linolenic, linoleic, and oleic. Useful forms of this oil can be characterized by the ratio of alpha linolenic:linoleic:oleic. The degradation rate of flax seed oil can be adjusted by altering the alpha linolenic:linoleic:oleic ratio, e.g., having a molar ratio of about 65:5:30, about 65:20:15, about 55:15:30, or about 55:20:25.

The nanoemulsion formulations include an oil phase of saturated fatty acid, monounsaturated fatty acid or PUFA rich oils that are biocompatible and/or biodegradable.

Oil compositions suitable for use as the oil phase of the nanoemulsion formulations according to the present disclosure can be from any source rich in mono-saturated or polyunsaturated fatty acids, such as plant or animal sources. Chemically or enzymatically derivatized, or completely synthetic, monounsaturated or polyunsaturated fatty acids are included within the scope of suitable components for the oil phase of the nanoemulsion formulations of the present disclosure. The concentration of the mono-unsaturated or polyunsaturated fatty acid in the oil phase can range from about 2% to about 100% (w/w), from about 5% to about 100% (w/w), or greater than 10% from about 20% to about 80% (w/w). The concentration of the oil phase, in the nanoemulsion formulation can vary from about 5% to about 40% (w/w), or from about 5% to about 30% (w/w). The concentration of hydrophobic chemotherapeutic agent soluble in the oil phase can range from about 0.01% to about 90% (w/w), from about 0.1% to about 45% (w/w), or greater than 0.5%, or from about 1% to about 30% (w/w). For example, the oils may contain high concentrations of mono-saturated or polyunsaturated fatty acids such as a concentration of greater than or equal to 10% (w/w) of at least one mono-unsaturated or polyunsaturated fatty acid of the omega-3, omega-6 or omega-9 family. A useful oil is one that can solubilize high concentrations of a hydrophobic chemotherapeutic agent including a Pt(II) complex. For example, useful oils are those containing high concentrations of linolenic or linoleic acid, e.g., oils of flax seed oil, black currant oil, pine nut oil or borage oil, and fungal oils such as spirulina and the like, alone or in combination.

C. Aqueous Phase

The aqueous phase of the nanoemulsion formulations according to the disclosure is purified and/or ultrapure water. This aqueous phase can also contain isotonicity modifiers such as, but not limited to, glycerine, low molecular weight polyethylene glycol (PEG), sorbitol, xylitol, or dextrose. The aqueous phase can alternatively or also contain pH adjusting agents such as, but not limited to, sodium hydroxide, hydrochloric acid, free fatty acids (oleic acid, linoleic acid, stearic acid, palmitic acid) and their sodium and potassium salts, preservative parabens, such as, but not limited to, methyl paraben or propyl paraben; antioxidants such as, but not limited to, ascorbic acid, α-tocopherol, and/or butylated hydroxy anisole. The concentration of the aqueous phase in the present nanoemulsion formulations can vary from 30% to 90% (w/w).

D. Interfacial Surface Membrane

The term “interfacial surface membrane” as used herein applies to the interface of the oil and aqueous phase and may refer either to a single pure emulsifier or a mixture of different emulsifiers and/or a mixture of emulsifiers and other components, such as stealth agents present in the interfacial surface membrane of the nanoemulsion formulation. The interfacial surface membrane or corona can comprise degradable lipids or emulsifiers bearing neutral, cationic and/or anionic side chains. The average surface area of the interfacial surface membrane corona on the nanoemulsion formulations described herein from may range from 30,000 nm² to 600,000 nm².

The interfacial surface may comprise an emulsifier and/or a stabilizer (stealth agent).

(1) Emulsifiers

The emulsifiers form part of the interface between the hydrophobic or oil core and the aqueous phase.

The emulsifiers comprise individual amphiphilic lipids and/or amphiphilic polymers. At least one emulsifier is present at the interface between the oil phase and the aqueous phase. The emulsifier can be an amphiphilic molecule such as a nonionic and ionic amphiphilic molecule. For example, the emulsifier can consist of neutral, positively-charged, or negatively-charged, natural or synthetic phospholipids molecules such as, but not limited to, natural phospholipids including soybean lecithin, egg lecithin, phosphatidylglycerol, phosphatidylinositol, phosphatidylethanolamine, phosphatidic acid, sphingomyelin, diphosphatidylglycerol, phosphatidylserine, phosphatidylcholine and cardiolipin; synthetic phospholipids including dimyristoylphosphatidylcholine, dimyristoylphosphatidyl ethyl-N-dimethyl propyl ammonium hydroxide, dimyristoylphosphatidylglycerol, distearoylphos-phatidylglycerol and dipalmitoylphosphatidylcholine; and hydrogenated or partially hydrogenated lecithins and phospholipids, e.g., from a natural source are used. The concentration of amphiphilic lipid in the nanoemulsion formulations can vary from about 0.5% to about 15% (w/v), or from about 1% to about 10% (w/v).

One nonlimiting example of a nanoemulsion formulation of the present disclosure comprises an oil and amphiphilic compounds of the interfacial surface membrane which surround or are dispersed within the oil and which form a continuous or discontinuous monomolecular layer. The interfacial surface membrane lowers the interfacial tension between the oil and aqueous phases, thereby enhancing the stability of the dispersed oil droplets in the surrounding aqueous phase. Further, the interfacial surface membrane of the nanoemulsion formulation localizes drugs, thereby providing therapeutic advantages by releasing the encapsulated chemotherapeutic drug, such as an hydrophobic Pt complex at predetermined, appropriate times.

An amphiphilic compound may have a polar head attached to a long hydrophobic tail. The polar portion is soluble in water, while the non-polar portion is insoluble in water. In addition, the polar portion may have either a formal positive charge, or a formal negative charge. Alternatively, the polar portion may have both a formal positive and a negative charge, and be a zwitterion or inner salt. Exemplary amphiphilic compounds include, for example, one or a plurality of the following: naturally derived lipids, surfactants, or synthesized compounds with both hydrophilic and hydrophobic moieties.

Specific examples of amphiphilic compounds making up a representative emulsifier include phospholipids, such as 1,2 distearoyl-sn-glycero-3-phosphoethanolamine (DSPE), dipalmitoylphosphatidylcholine (DPPC), distearoylphosphatidylcholine (DSPC), diarachidoylphosphatidylcholine (DAPC), dibehenoylphosphatidylcholine (DBPC), ditricosanoylphosphatidylcholine (DTPC), and dilignoceroylphatidylcholine (DLPC), incorporated at a ratio of about 0.5% to about 2.5% (weight lipid/w oil), about between 1.0% to about 1.5% (weight lipid/w oil). Phospholipids, which may be used, include, but are not limited to, phosphatidic acids, phosphatidyl cholines with both saturated and unsaturated lipids, phosphatidyl ethanolamines, phosphatidylglycerols, phosphatidylserines, phosphatidylinositols, lysophosphatidyl derivatives, cardiolipin, and β-acyl-y-alkyl phospholipids. Examples of phospholipids include, but are not limited to, phosphatidylcholines such as dioleoylphosphati-dylcholine, dimyristoylphosphatidylcholine, dipentadecanoylphosphatidylcholine dilauroylphos-phatidylcholine, dipalmitoylphosphatidylcholine (DPPC), distearoylphos-phatidylcholine (DSPC), diarachidoylphosphatidylcholine (DAPC), dibehenoylphosphatidylcho-line (DBPC), ditricosanoylphosphatidylcholine (DTPC), dilignoceroylphatidylcholine (DLPC); and phosphatidylethanolamines such as dioleoylphosphatidylethanolamine or 1-hexadecyl-2-palmitoylglycerophos-phoethanolamine. Synthetic phospholipids with asymmetric acyl chains (e.g., with one acyl chain of 6 carbons and another acyl chain of 12 carbons) may also be used.

An amphiphilic compound of the interfacial membrane may include lecithin or phosphatidylcholine.

(2) Stabilizers

The stabilizer or stealth agent may or may not be part of the interfacial surface membrane. If it is a part of this membrane, it can be added with the emulsifier when preparing a nanoemulsion formulation of the present disclosure. The stabilizer may be an amphilic molecule.

One representative stabilizer is a PEGylated lipid. Some useful phospholipid molecules are natural phospholipids including polyethylene glycol (PEG) repeat units, which can also be referred to as a “PEGylated” lipid or lipidated PEG. Such PEGylated lipids can control inflammation and/or immunogenicity (i.e., the ability to provoke an immune response) and/or lower the rate of clearance from the circulatory system via the reticuloendothelial system (RES) and/or the mononuclear phagocyte system (MPS), due to the presence of the poly(ethylene glycol) groups, PEGylated soybean lecithin, PEGylated egg lecithin, PEGylated phosphati-dylglycerol, PEGylated phosphatidylinositol, PEGylated phosphatidylethanolamine, PEGylated phosphatidic acid, PEGylated sphingomyelin, PEGylated diphosphatidylglycerol, PEGylated phosphatidylserine, PEGylated phosphatidylcholine and PEGylated cardiolipin; synthetic phospholipids including PEGylated dimyristoylphosphatidylcholine, PEGylated dimyristoylphosphatidylglycerol, PEGylated distearoylphosphatidylglycerol and PEGylated dipalmitoylphosphatidylcholine; and hydrogenated or partially hydrogenated PEGylated lecithins and PEGylated phospholipids. Such amphiphilic PEGylated lipids can be used alone or in combination. The concentration of amphiphilic PEGylated lipid in the nanoemulsions can vary from about 0.01% to 15% (w/v), or from about 0.05% to 10% (w/v).

Exemplary lipids that can be part of the PEGylated lipid include, but are not limited to, fatty acids such as long chain (e.g., C8-050), substituted, or unsubstituted hydrocarbons. A fatty acid group can be a C10-C20 fatty acid or salt thereof, a C15-C20 fatty acid or salt thereof, or a fatty acid can be unsaturated, monounsaturated, or polyunsaturated. For example, a fatty acid group can be one or more of butyric, caproic, caprylic, capric, lauric, myristic, palmitic, stearic, arachidic, behenic, lignoceric, palmitoleic, oleic, vaccenic, linoleic, alpha-linolenic, gamma-linoleic, arachidonic, gadoleic, arachidonic, eicosapentaenoic, docosahexaenoic, or erucic acid.

Other exemplary stabilizers are phosphatide, a polyglycerol mono oleate, PEG₂₀₀₀DSPE, PEG₃₄₀₀DSPE, PEG₅₀₀₀DSPE, or any combination thereof. Useful stabilizers are a PEG derivative, a phosphatide, and/or polyglycerol mono oleate and useful non-limiting PEG derivatives are PEG₂₀₀₀DSPE, PEG₃₄₀₀DSPE, PEG₅₀₀₀DSPE.

The PEGylation density may be varied as necessary to facilitate long-circulation in the blood (Perry et al. (2012) Nano. Lett. 12:5304-5310). In some cases, the addition of PEG repeat units may increase plasma half-life of the nanoemulsion formulation, for instance, by decreasing the uptake of the nanoemulsion formulation by the MPS, while decreasing transfection/uptake efficiency by cells. Those of ordinary skill in the art will know of methods and techniques for PEGylating a lipid, for example, by using EDC (1-ethyl-3-(3-dimethylaminopropyl) carbodiimide hydrochloride) and NHS (N-hydroxysuccinimide) to react a lipid that will be in the corona of the nanoemulsion formulation to a PEG group terminating in an amine, by ring opening polymerization techniques (ROMP), or the like.

PEG may include a terminal end group, for example, when PEG is not conjugated to a ligand. For example, PEG may terminate in a hydroxyl, a methoxy or other alkoxyl group, a methyl or other alkyl group, an aryl group, a carboxylic acid, an amine, an amide, an acetyl group, a guanidino group, or an imidazole. Other contemplated end groups include azide, alkyne, maleimide, aldehyde, hydrazide, hydroxylamine, alkoxyamine, or thiol moieties.

The molecular weight of the PEG on interfacial membrane surface of the nanoemulsion formulation can be optimized for effective treatment as disclosed herein. For example, the molecular weight of a PEG may influence particle degradation rate (such as adjusting the molecular weight of a biodegradable PEG), solubility, water uptake, and drug release kinetics. For example, the molecular weight of the PEG can be adjusted such that the particle biodegrades in the subject being treated within a period of time ranging from a few hours, to 1 to 2 weeks, to 3 to 4 weeks, to 5 to 6 weeks, to 7 to 8 weeks, etc. One useful nanoemulsion formulation comprises a copolymer PEG conjugated to a lipid, the PEG having a molecular weight of about 1 kD to about 20 kD, about 5 kD to about 20 kD, or about 10 kD to about 20 kD, and the lipid can have a molecular weight of about 200 Da to about 3 kD, about 500 Da to about 2.5 kD, or about 700 Da to about 1.5 kD. An exemplary nanoemulsion formulation includes about 5 weight percent to about 30 weight percent monounsaturated or polyunsaturated fatty acid rich oil, or about 0.5 weight percent to about 5 weight percent primary emulsifier, or about 0.1 weight percent to about 1.0 weight percent co-emulsifiers or about 0.1 to about 0.75 weight percent, PEG-derivatives. Exemplary lipid-PEG copolymers can include a number average molecular weight of about 1.5 kD to about 25,000 kDa, or about 2 kD to about 20 kD.

The ratio of oil to emulsifier to stabilizer in the nanoemulsion formulation for example, flax seed oil to emulsifier to PEGylated lipid stabilizer, may be selected to optimize certain parameters such as size, chemotherapeutic agent release, and/or nanoemulsion formulation degradation kinetics.

An alternative stabilizer may contain poly(ester-ether)s. For example, the interfacial membrane surfaces of the nanoemulsion formulation can have repeat units joined by ester bonds (e.g., R—C(O)-O—R′ bonds) and ether bonds (e.g., R—O—R′ bonds). A biodegradable component of the interfacial membrane surface of the nanoemulsion formulation, such as a hydrolyzable biopolymer containing carboxylic acid groups, may be conjugated with poly(ethylene glycol) repeat units to form a poly(ester-ether) coating on the interfacial membrane surface of the nanoemulsion formulation.

E. Targeting Ligands

The nanoemulsion formulation of the present disclosure may further comprise a targeting ligand or molecule which is specific for a receptor or protein or molecule on the cancer cells to be treated or imaged. In that way the nanoemulsion formulation is delivered more accurately to the cells having the target, such as targeting ligand receptors that are found in greater amounts on cancer cells than on normal cells.

One such useful target is epidermal growth factor receptor (EGFR) (see, e.g., Magadala et al. (2008) AAPS. J. 10:565-576). EGFR is a member of the human epidermal growth factor receptor HER/erb family of receptor tyrosine kinases, which plays important roles in both cell growth and differentiation. Overexpression of EGFR is associated negatively with progression-free and overall survival in a wide variety of human cancers, including lung, breast, bladder, and ovarian cancers. Its positive signaling causes increased proliferation, decreased apoptosis, and enhanced tumor cell motility and angiogenesis.

Useful EGFR-targeting ligands include, but are not limited to, the amino acid peptide Y-W-Y-G-Y-T-P-Q-N-V-I (SEQ ID. NO:1, peptide 4) or an anti-EGFR immunoglobulin, e.g., a nanobody such as EGa1-PEG.

Another useful target is the folate receptor. Folate receptor alpha (FR-α) is one of three isoforms: α, β, & γ. FR-α is a 38 kD glycosyl-phosphatidylinositol-anchored glycoprotein that binds folic acid (and internalizes it) with a Kd of less than 1 nM, and is highly expressed in a number of human tumors including ovarian (>85%), lung (>75%), breast (>60%) renal cell (>65%), brain, head, and neck. (Fisher et al. (2008) J. Nucl. Med. 49:899-906). In normal tissue its expression is much lower and limited to kidney tubuli, lung epithelium in the apical cell, the choroid plexus, and placenta. FR-α over expression is negatively associated with overall survival in ovarian and other cancers. However, with over 85% of ovarian tumors expressing FR-α it is difficult to correlate expression with mortality. As a predictor of response rate to chemotherapy, complete or partial remission, patients with FR-α greater than median levels had a 15 times higher likelihood of negative response.

Useful folate-targeting ligands include, but are not limited to, 1,2-distearoyl-sn-glycero-3-phosphoethanolamine-N-[methoxy(polyethylene glycol)-2000]-cysteine-folic acid (DSPE-PEG-cysteine-folic acid), 1,2-distearoyl-sn-glycero-3-phosphoethanolamine-N-[methoxy (polyethylene glycol)-3400]-folic acid, 1,2-distearoyl-sn-glycero-3-phosphoethanolamine-N-[folate(polyethylene glycol)-2000] (ammonium salt) (DSPE-PEG(2000) folate), 1,2-distearoyl-sn-glycero-3-phosphoethanolamine-N-[folate(polyethylene glycol)-5000] (ammonium salt) (DSPE-PEG(5000) folate) (Avanti Polar Lipids, Inc. Alabaster, Ala.), and any combinations thereof.

In some nanoemulsion formulations, the targeting moieties are attached, e.g., covalently bonded, to a lipid component of the nanoemulsion formulation. One exemplary nanoemulsion formulation comprises a Pt(II) complex, an oil core comprising functionalized and non-functionalized oils, an interfacial surface membrane or corona, and a low-molecular weight targeting ligand, wherein the targeting ligand is covalently bonded, to the lipid component of the nanoemulsion formulation's interfacial surface membrane.

F. Imaging Moieties

Nanoemulsion formulations of the present disclosure can further include imaging or contrast agents. The use of imaging agents on the nanoemulsion formulation of the present disclosure allows physicians to track in real time the amount of chemotherapeutic agent actually reaching the site of disease. Physicians can then quickly decide whether a particular patient should continue with treatment. Useful imaging agents include paramagnetic agents such as gadolinium (Gd), iron oxide, iron platinum, and manganese. Useful gadolinium derivatives include 1,2-dimyristoyl-sn-glycero-3-phosphoethanolamine-N-diethylene-triaminepentaacetic acid (Gd-DTPA-PE), 1,2-dimyristoyl-sn-glycero-3-phosphoethanolamine-N-1,4,7,10-tetraazacyclododecane-1,4,7,10-tetraacetic acid (Gd-DOTA-PE), and 1,2-dimyristoyl-sn-glycero-3-paraazoxyphenetole-N-1,4,7,10-tetraazacyclododecane-1,4,7,10-tetraacetic acid (Gd-PAP-DOTA) (Avanti Polar Lipids, Inc. Alabaster, Ala.). These gadolinium-based MRI contrast moieties can be prepared or obtained and incorporated into a nanoemulsion formulation as described herein. Useful imaging agents are gadolinium, iron oxide, iron, platinum, and manganese. Examples of suitable gadolinium imaging agents are Gd-DTPA-PE, Gd-DOTA-PE, Gd-PAP-DOTA.

Accordingly, a representative nanoemulsion formulation comprises an imaging moiety attached, e.g., covalently bonded, to a lipid component of the nanoemulsion formulation. One exemplary nanoemulsion formulation comprises a therapeutic Pt(II) complex, an oil core comprising functionalized and non-functionalized oils, an interfacial surface membrane or corona, an EGFR targeting ligand, and an imaging agent, wherein the imaging agent is covalently bonded to the lipid component of the nanoemulsion formulation's interfacial surface membrane.

In another exemplary nanoemulsion formulation, imaging moieties are soluble in the oil phase. For example, a nanoemulsion formulation comprises a chemotherapeutic Pt(II) complex, an oil phase comprising functionalized and non-functionalized oils, an interfacial surface membrane, an EGFR targeting ligand, and an imaging agent, wherein the imaging agent is soluble in the oil phase.

### 2. Preparation of Nanoemulsion Formulations

The nanoemulsion formulations of the present disclosure can be prepared from various intermediates and component constituents, for example, as described in Examples 5-11 below, and can be made using a microfluidizer (Microfluidics Corp., Newton, Mass.).

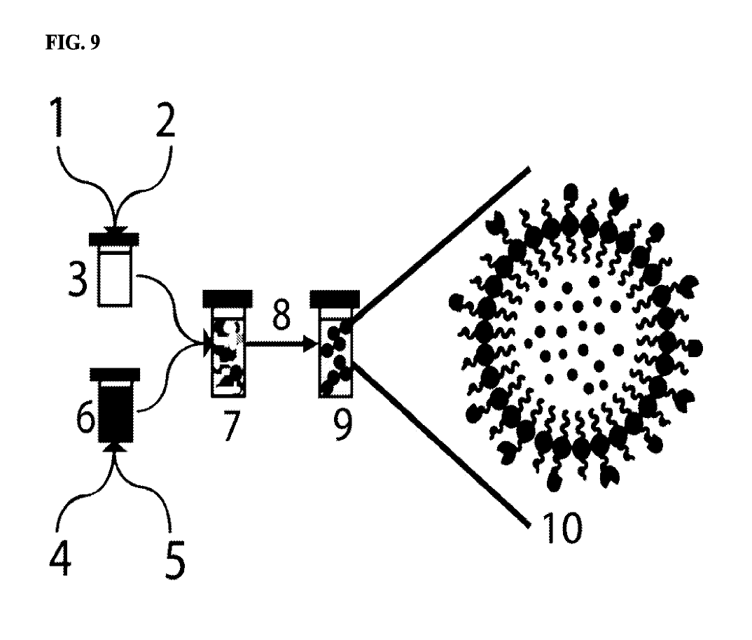

FIG. 9 shows a representative synthesis scheme for one non-limiting, EGFR-targeted, Gd-labeled nanoemulsion formulation of the present disclosure. In this figure, 1 is a platinum mono-fatty acid derivative, where R₁ and R₂ are NH₃ groups and R₃ is fatty acid with a carbon chain length of C14, C16, or C18. 2 is C6-ceramide, a proapoptotic agent. 3 represents the compounds 1 and 2 being dissolved in chloroform and added to flax seed oil. Chloroform is removed using nitrogen, resulting in oil phase formation. 4 is the imaging moiety Gd-DTPA-PE. 5 is the targeting ligand EGFR_{BP}-PEG-DSPE. 6 represents the compounds of 4 and 5 being added to egg lecithin and PEG₂₀₀₀DSPE in glycerol water solution, resulting in aqueous phase formation. 7 represents the oil phase of 3 and aqueous phase of 6 being combined, mixed and heated to 60° C. for 5 minutes to form the coarse emulsion. 8 represents the coarse emulsion of 7 being emulsified using a high pressure homogenizer (LV1 Microfluidizer) at 25,000 psi for 10 cycles to obtain nanoemulsion formulation droplets of a size below 150 nm. 9 is the resulting nanoemulsion formulation of one embodiment of the present disclosure with a size below 150 nm. 10 is a representative drawing of an individual resulting nanoemulsion formulation.

Nanoemulsion formulations were prepared with a concentration of up to 5 mg/ml of Pt-MMA, Pt-MPA, Pt-MSA, Pt-SA.

### 3. Characterization of Nanoemulsion Formulations

The nanoemulsion formulations of the present disclosure may have a substantially spherical shape. For instance, the nanoemulsion formulations generally appear to be spherical, or non-spherical configuration, but upon shrinkage, may adopt a non-spherical configuration. These nanoemulsion formulations may have a characteristic dimension of less than about 1 μm, where the characteristic dimension of a nanoemulsion formulation is the diameter of a perfect sphere having the same volume as the nanoemulsion formulation. For example, the characteristic dimensions of the nanoemulsion formulation can be less than about 300 nm, less than about 200 nm, less than about 150 nm, less than about 100 nm, or less than about 50 nm in some cases. Some disclosed nanoemulsion formulations may have a diameter of about 50 nm to 200 nm, about 50 nm to about 180 nm, about 80 nm to about 160 nm, or about 80 nm to about 150 nm.

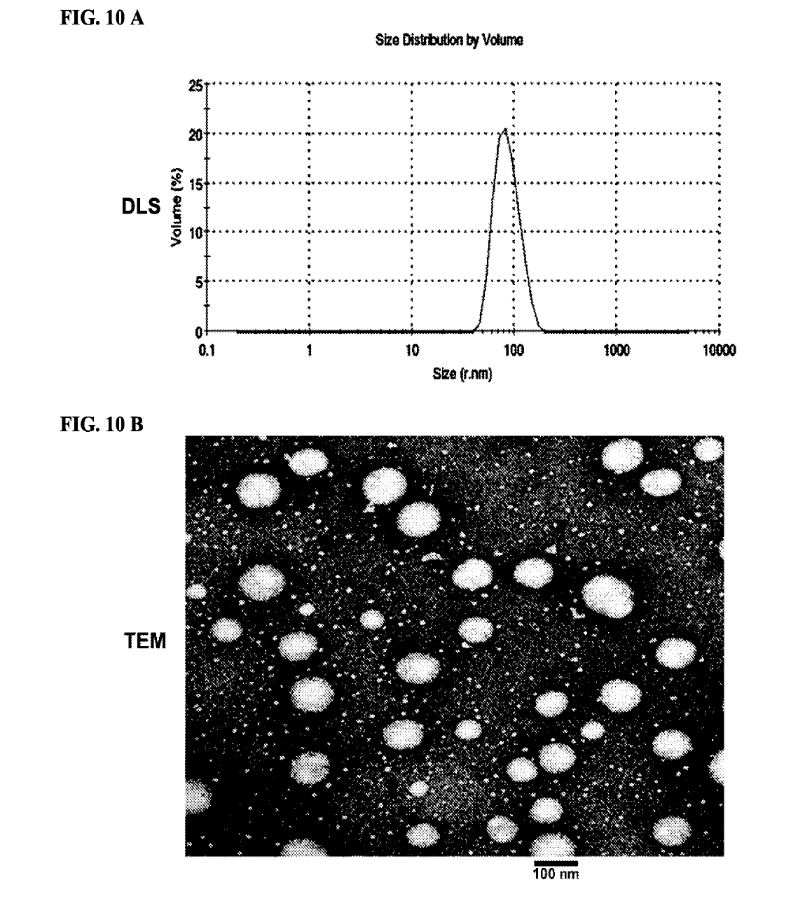

The size of the particles in the nanoemulsion formulation was determined by dynamic light scattering (DLS) (Zetasizer ZS, Malvern Instruments Ltd., Worcestershire, United Kingdom) (FIG. 10A) and by transmission electron microscopy (TEM) (FIG. 10B). The particle size of this representative nanoemulsion formulation was below 150 nm in diameter.

Nanoemulsion formulation size distribution and zeta potential values of control blank nanoemulsion formulation, EGFR-targeted blank nanoemulsion formulation and EGFR-targeted nanoemulsion formulations were determined using Zetasizer ZS (Malvern Instruments, Worcestershire, United Kingdom). The results are shown below in Table IV.

TABLE IV

| | Hydrodynamic radius | | Zeta Potential |
|---|---|---|---|
| | of nanoemulsion droplets | | (mV) |
| NE Formulation | Avg (nm) ± SD | Polydispersity Index | Avg ± SD |
| Blank NE | 89.6 ± 5.0 | 0.06 ± 0.03 | −55.6 ± 0.4 |
| Blank NE- | 70.0 ± 0.8 | 0.04 ± 0.03 | −49.1 ± 10.0 |
| EGFR Targeted | | | |
| Pt-MMA NE | 88.0 ± 0.3 | 0.07 ± 0.03 | −38.4 ± 9.6 |
| EGFR Targeted | | | |
| Pt-MPA NE | 67.0 ± 11.0 | 0.07 ± 0.01 | −43.0 ± 15.0 |
| EGFR Targeted | | | |
| Pt-MSA NE | 73.8 + 1.0 | 0.06 + 0.02 | −39.3 + 9.6 |
| EGFR Targeted | | | |
| Pt-SA NE | 71.0 ± 5.0 | 0.10 + 0.01 | −48.5 ± 10.0 |
| EGFR Targeted | | | |

The average particle size of the control nanoemulsion formulation containing no Pt(II) complex, CER or targeting moiety was below 200 nm in diameter. The incorporation of Pt(II) complex, CER and targeting moiety in the nanoemulsion formulations did not significantly change the hydrodynamic particle size and size remained below 200 nm. The average surface charges of the nanoemulsion formulations were in the range of −38 mV to −56 mV.

Nanoemulsion formulation size distribution of control blank nanoemulsion formulation, and EGFR-targeted nanoemulsion formulations were determined using Zetasizer ZS (Malvern Instruments, Worcestershire, United Kingdom) at 4° C. and room temperature (RT) for up to three month. The results are shown in below Table V.

TABLE V
Particle Size (nm) and polydispersity index (in parentheses) during storage

| | | 15 days | | 1 Month | | 3 Months | |
|---|---|---|---|---|---|---|---|
| NE Formulation | 0 days | 4° C. | 25° C. | 4° C. | 25° C. | 4° C. | 25° C. |
| Blank NE | 70 ± 0.8 | 70 ± 2 | 70 ± 1 | 70 ± 1 | 69 ± 2 | 143 ± 1.2 | 130 ± 0.5 |
| | (0.04) | (0.10) | (0.03) | (0.10) | (0.10) | (0.09) | (0.10) |
| Pt-MMA NE | 88 ± 0.3 | 89 ± 3 | 79 ± 2 | 86 ± 3 | 96 ± 2 | 173 ± 7 | 168 ± 2 |
| EGFR Targeted | (0.07) | (0.10) | (0.03) | (0.10) | (0.10) | (0.10) | (0.10) |
| Pt-MPA NE | 67 ± 11 | 68 ± 1 | 102 ± 2 | 67 ± 1 | 67 ± 2 | 140 ± 5 | 140 ± 1 |
| EGFR Targeted | (0.07) | (0.06) | (0.05) | (0.10) | (0.10) | (0.14) | (0.10) |
| Pt-MSA NE | 73 ± 0.5 | 79 ± 3.5 | | 77 ± 5.5 | | | |
| EGFR Targeted | (0.04) | (0.10) | | (0.10) | | | |
| Pt-SA NE | 71 ± 2 | 70 ± 2 | 102 ± 1 | 72 ± 2 | 100 ± 2 | 169 ± 8 | 160 ± 5 |
| EGFR Targeted | (0.10) | (0.10) | (0.10) | (0.10) | (0.10) | (0.10) | (0.10) |

The average particle size of the control nanoemulsion formulation containing no Pt(II) complex, CER or targeting moiety remained below 200 nm in diameter for up to 3 months. The incorporation of Pt(II) complex, CER and targeting moiety in the nanoemulsion formulations did not significantly change the hydrodynamic particle size and size remained below 200 nm for up to 3 months indicating that the nanoemulsion formulations were stable at both 4° C. and RT for up to 3 months.

The nanoemulsion formulations of the present disclosure may have an interior and a surface, where the surface has a composition different from the interior, i.e., there may be at least one compound present in the interior but not present on the surface (or vice versa), and/or at least one compound is present in the interior and on the surface at differing concentrations. For example, a compound, such as a targeting moiety (i.e., a low-molecular weight ligand, protein, carbohydrate, or nucleic acid) of a polymeric conjugate of the present disclosure, may be present in both the interior and the surface of the nanoemulsion formulation, but at a higher concentration on the surface than in the interior of the nanoemulsion formulation, although in some cases, the concentration in the interior of the nanoemulsion formulation may be essentially nonzero, i.e., there is a detectable amount of the compound present in the interior of the nanoemulsion.

In some cases, the interior of the nanoemulsion formulation is more hydrophobic than the surface of the nanoemulsion formulation. For instance, the interior of the nanoemulsion formulation may be relatively hydrophobic with respect to the surface of the nanoemulsion formulation, and a drug or other payload may be hydrophobic, and readily associates with the relatively hydrophobic center of the nanoemulsion formulation. The drug or other payload can thus be contained within the interior of the nanoemulsion formulation, which can shelter it from the external environment surrounding the nanoemulsion formulation (or vice versa). For instance, a drug or other payload contained within a nanoemulsion formulation administered to a subject will be protected from a subject's body, and the body may also be substantially isolated from the drug for at least a period of time.

For example, an exemplary nanoemulsion formulation may have a PEG derivative corona with a density of about 1.065 g/cm³, or about 1.01 g/cm³ to about 1.10 g/cm³.

The nanoemulsion formulations of the present disclosure may have controlled release properties, e.g., may be capable of delivering an amount of active agent to a patient, for example to a specific site in a patient, over an extended period of time, for example over 1 day, 1 week, or more. Some disclosed nanoemulsion formulations substantially immediately release (for example over about 1 minute to about 30 minutes), less than about 2% in 6 hours, less than about 4% in 24 hours, less than about 7% in 48 hours, or less than about 10% of a chemotherapeutic agent (for example Pt(II) complex) in 72 hours, for example when placed in a phosphate buffer saline solution at room temperature and/or at 37° C.

### 4. Method of Treatment

The nanoemulsion formulation in accordance with the present disclosure may be used to treat, alleviate, ameliorate, relieve, delay onset of, inhibit progression of, reduce severity of, and/or reduce incidence of one or more symptoms or features of a cancer or tumor.

The term “cancer” includes pre-malignant as well as malignant cancers. Cancers include, but are not limited to, prostate, gastric cancer, colorectal cancer, skin cancer, e.g., melanomas or basal cell carcinomas, lung cancer, cancers of the head and neck, bronchus cancer, pancreatic cancer, urinary bladder cancer, brain or central nervous system cancer, peripheral nervous system cancer, esophageal cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, and the like. “Cancer cells” can be in the form of a tumor or exist alone within a subject (e.g., leukemia cells).

In certain cases, targeted nanoemulsion formulation may be used to treat any cancer where EGFR or folate is expressed on the surface of cancer cells or in the tumor neovasculature, including the neovasculature of ovarian or non-ovarian solid tumors. Examples of the EGFR- or folate-related indications include, but are not limited to, breast, ovarian, esophageal, and oropharyngeal cancers.

When treating cancer, a “therapeutically-effective amount” of the nanoemulsion formulation of the present disclosure is administered and is that amount effective for treating, alleviating, ameliorating, relieving, delaying onset of, inhibiting progression of, reducing severity of, and/or reducing incidence of one or more symptoms or features of the cancer.

As will be appreciated by those of ordinary skill in this art, the effective amount of the nanoemulsion formulation may vary depending on such factors as the desired biological endpoint, the drug to be delivered, the target tissue, the route of administration, etc. For example, the effective amount of the nanoemulsion formulation is the amount that results in a reduction in tumor size by a desired amount over a desired period of time. Additional factors which may be taken into account include the severity of the disease state; age, weight and gender of the patient being treated; diet, time and frequency of administration; reaction sensitivities; and tolerance/response to therapy.

The nanoemulsion formulations of the present disclosure can be used to inhibit the growth of cancer cells, e.g., ovarian cancer cells. As used herein, the term “inhibits growth of cancer cells” or “inhibiting growth of cancer cells” refers to any slowing of the rate of cancer cell proliferation and/or migration, arrest of cancer cell proliferation and/or migration, or killing of cancer cells, such that the rate of cancer cell growth is reduced in comparison with the observed or predicted rate of growth of an untreated control cancer cell. The term “inhibits growth” can also refer to a reduction in size or disappearance of a cancer cell or tumor, as well as to a reduction in its metastatic potential. Such an inhibition at the cellular level may reduce the size, deter the growth, reduce the aggressiveness, or prevent or inhibit metastasis of a cancer in a patient. Those skilled in the art can readily determine, by any of a variety of suitable indicia, whether cancer cell growth is inhibited.

Inhibition of cancer cell growth may be evidenced, for example, by arrest of cancer cells in a particular phase of the cell cycle, e.g., arrest at the G2/M phase of the cell cycle. Inhibition of cancer cell growth can also be evidenced by direct or indirect measurement of cancer cell or tumor size. In human cancer patients, such measurements generally are made using well known imaging methods such as magnetic resonance imaging, computerized axial tomography and X-rays. Cancer cell growth can also be determined indirectly, such as by determining the levels of circulating carcinoembryonic antigen, prostate specific antigen or other cancer-specific antigens that are correlated with cancer cell growth Inhibition of cancer growth is also generally correlated with prolonged survival and/or increased health and well-being of the subject.

Also provided herein are therapeutic protocols that include administering a therapeutically effective amount of a disclosed therapeutic nanoemulsion formulation to a healthy individual (i.e., a subject who does not display any symptoms of cancer and/or who has not been diagnosed with cancer). For example, healthy individuals may be “immunized” with an inventive targeted or non-targeted particle, such as a nanoemulsion formulation, prior to development of cancer and/or onset of symptoms of cancer; at risk individuals (for example, patients who have a family history of cancer; patients carrying one or more genetic mutations associated with development of cancer; patients having a genetic polymorphism associated with development of cancer; patients infected by a virus associated with development of cancer; patients with habits and/or lifestyles associated with development of cancer; etc.) can be treated substantially contemporaneously with (for example, within 48 hours, within 24 hours, or within 12 hours of) the onset of symptoms of cancer. Individuals known to have cancer may receive inventive treatment at any time.

Nanoemulsion formulations disclosed herein may be combined with pharmaceutical acceptable carriers to form a pharmaceutical composition. As would be appreciated by one of skill in this art, the carriers may be chosen based on the route of administration as described below, the location of the target issue, the drug being delivered, the time course of delivery of the drug, etc.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parentally acceptable diluent or solvent, for example, as a solution in 1,3-butanediol. Among the acceptable vehicles and solvents that may be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono or di-glycerides. In addition, fatty acids such as oleic acid are used in the preparation of injectables.

### 5. Methods of Administration of Nanoemulsion Formulations

The nanoemulsion formulations of this disclosure can be administered to a patient by any means known in the art including oral and parenteral routes. The term “patient,” as used herein, refers to humans as well as non-humans, including, for example, mammals, birds, reptiles, amphibians, and fish. Sometimes parenteral routes are chosen since they avoid contact with the digestive enzymes that are found in the alimentary canal. These compositions may be administered by injection (e.g., intravenous, subcutaneous or intramuscular, intraperitoneal injection), rectally, vaginally, topically (as by powders, creams, ointments, or drops), or by inhalation (as by sprays).

For example, the nanoemulsion formulations of the present disclosure may be administered to a subject in need thereof systemically, e.g., by intravenous infusion or injection. Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parentally acceptable diluent or solvent, for example, as a solution in 1,3-butanediol. Among the acceptable vehicles and solvents that may be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono or di-glycerides. In addition, fatty acids such as oleic acid are used in the preparation of injectables.

The nanoemulsion formulations may also be administered orally. Solid dosage forms for oral administration include capsules, tablets, pills, powders, and granules. In such solid dosage forms, the encapsulated or unencapsulated conjugate is mixed with at least one inert, pharmaceutically-acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar-agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may also comprise buffering agents.

It will be appreciated that the exact dosage of the nanoemulsion formulation of the present disclosure is chosen by the individual physician in view of the patient to be treated, in general, dosage and administration are adjusted to provide an effective amount of the nanoemulsion formulation to the patient being treated. As used herein, the “effective amount” of a nanoemulsion formulation refers to the amount that elicits the desired biological response. As will be appreciated by those of ordinary skill in this art, the effective amount of the nanoemulsion formulations may vary depending on such factors as the desired biological endpoint, the drug to be delivered, the target tissue, the route of administration, etc. For example, the effective amount of the nanoemulsion formulation is the amount that results in a reduction in tumor size by a desired amount over a desired period of time. Additional factors which may be taken into account include the severity of the disease state; age, weight and gender of the patient being treated; diet, time and frequency of administration; reaction sensitivities; and tolerance/response to therapy

The nanoemulsion formulation of the present disclosure may be formulated in dosage unit form for ease of administration and uniformity of dosage. The expression “dosage unit form” as used herein refers to a physically discrete unit of the nanoemulsion formulation appropriate for the patient to be treated. It will be understood, however, that the total daily usage of the nanoemulsion formulations of the present disclosure will be decided by the attending physician within the scope of sound medical judgment. For any nanoemulsion formulation, the therapeutically-effective dose can be estimated initially either in cell culture assays or in animal models, usually mice, rabbits, dogs, or pigs. The animal model is also used to achieve a desirable concentration range and route of administration. Such information can then be used to determine useful doses and routes for administration in humans. Therapeutic efficacy and toxicity of the nanoemulsion formulations can be determined by standard pharmaceutical procedures in cell cultures or experimental animals, e.g., ED₅₀ (the dose is therapeutically effective in 50% of the population) and LD₅₀ (the dose is lethal to 50% of the population). The dose ratio of toxic to therapeutic effects is the therapeutic index, and it can be expressed as the ratio, LD₅₀/ED₅₀ Nanoemulsion formulations, which exhibit large therapeutic indices may be useful in some embodiments. The data obtained from cell culture assays and animal studies can be used in formulating a range of dosage for human use.

For example, the nanoemulsion formulation may contain platinum compounds at a concentration of about 0.001% to about 2% (about 0.01 mg/ml to about 20 mg/ml). The dosage administered by injection may contain platinum in the range of about 5 mg to about 1000 mg in the first day of every 1 to 4 weeks depending upon the patient. One might administer a dosage of about 50 mg to about 400 mg in the first day of every 1 to 4 weeks to a patient having a body weight of about 40 kg to about 100 kg. Such dosages may prove useful for patients having a body weight outside this range. The nanoemulsion formulation may also contain C₆-ceramide that acts as a proapoptotic agent, and enhances platinum cytotoxicity in the cancer cells. The concentration of C₆-ceramide in the composition is about 0.001% to about 2% (about 0.01 mg/ml to about 20 mg/ml).

Emulsion for oral administration are of about the same volume as those used for injection. However, when administering the drug orally, higher doses may be used when administering by injection. For example, a dosage containing about 10 mg to about 1500 mg platinum in the first day of every 1 to 4 weeks may be used. In preparing such liquid dosage form, standard making techniques may be employed.

### 6. Imaging Methods

The nanoemulsion formulations in accordance with the present disclosure may be used to image tumors or cancer cells. These nanoemulsion formulations are small enough to travel into minute body regions and, when coupled with paramagnetic elements, such as gadolinium ions (Gd³⁺), iron oxide, iron, platinum, or manganese, can enhance tissue contrast in MRI. Once the nanoemulsion formulation has reached the cancer site, its efficacy is determined, which can be done using an in vivo imaging modality such as MRI. Image-guided therapy using nanoemulsion formulations couples drug delivery with tissue imaging to allow clinicians to efficiently deliver chemotherapeutic agents, while simultaneously localizing the drugs and visualizing their physiological effects.

The nanoemulsion formulations combined with an appropriate imaging agent can act as MRI contrast agents to enhance tissue image resolution. Contrast agents such as Gd³⁺ have unpaired electrons that interact with surrounding water molecules to decrease their proton spin time, also referred to as T₁. Relaxation time is defined as the period it takes for a proton to return to its equilibrium position following a magnetization pulse. MRI can measure T₁ by creating a magnetic field that reverses the sample's magnetization, and then recording the time required for the spin directions to realign in their equilibrium positions again. The decreased T₁ relaxation time of the target tissue allows an MRI machine to better distinguish between it and its surrounding aqueous environment.

The nanoemulsion formulations according to the disclosure can serve as a new Gd³⁺ chelated, EGFR- or folate receptor-targeted nanoemulsion formulation that not only exhibits MRI contrast but also carries encapsulated Pt(II) complexes or Pt(II) complexes and a chemopotentiator to the target tissue for successful image-guided therapy. To examine the MRI contrast potential of these nanoemulsions, in vivo studies were conducted using MRI, while cell uptake and trafficking as well as efficacy studies were conducted to examine the drug delivery potential of the nanoemulsion formulation.

The method of imaging includes administering to a patient or subject to be imaged a diagnostically effective amount of a nanoemulsion formulation according to the disclosure. The nanoemulsion formulation can be administered by a variety of techniques including subcutaneously and intravenously. The method is effective for imaging cancers, such as breast, ovarian, esophageal, and oropharyngeal cancers and other cancers accessible by the lymphatic or vascular (blood) systems. For magnetic resonance imaging methods, the nanoemulsion formulation of the disclosure includes a paramagnetic metal ion (e.g., Gd³⁺).

The following examples provide specific exemplary methods of the invention, and are not to be construed as limiting the invention to their content.

### EXAMPLES

### Example 1

### Synthesis of cis-Diamine Pt (II) Chloride Monopalmitic Acid (Pt-MPA)

The cisplatin intermediate was prepared as follows. Cis-dichlorodiamine Pt (II) (240 mg, 0.8 mmoles) (Sigma) was suspended in 30 ml distilled water and heated to 70° C. to dissolve the complex. The solution was then cooled to RT. Thereafter, an aqueous solution of silver nitrate ((135.9 mg, 0.8 mmoles) in 10 ml of water) was added drop-wise to the solution of starting material under stirring (400 rpm). The formation of translucent white precipitate of silver chloride was commenced immediately after the addition of the aqueous solution. The mixed solution was stirred for 3 hr at RT under light-shielding conditions. The resulting precipitate of silver chloride was filtered (Corning polystyrene Filter System, Corning (0.22 μM)) and washed with water. The combined filtrate was used in the following step without further treatment.

The Pt (II) monopalmitate complex was prepared as follows. An aqueous solution of sodium palmitate ((223 mg, 0.8 mmoles) (Sigma) in 10 ml of water) was added to the aqueous solution obtained above and stirred at RT for 3 wk under light-shielding conditions to complete the reaction there between. The translucent white precipitate formed was filtered off, washed with a small amount of ether, and dried in a vacuum desiccator to obtain the crude product. The crude product contained a mixture of mono- and di-fatty acid Pt derivatives. Because the mono-fatty acid Pt derivative was soluble in chloroform, chloroform was used to purify the mixture. The crude product was suspended in 25 ml chloroform in conical tube, vortex mixed for 5 min, and kept at RT for 24 hr. Tubes were then centrifuged (5000 rpm, 10 min) (Beckman-Coulter, Inc., Brea, Calif.), and the supernatant was transferred into glass vials and vacuum dried to obtain the Pt (II) monomyristic acid complex (yield=22%).

### Example 2

### Synthesis of cis-Diamine Pt (II) Chloride Monostearic Acid (Pt-MSA)

The cisplatin intermediate was prepared as follows. Cis-dichlorodiamine Pt (II) (240 mg, 0.8 mmoles) (Sigma) was suspended in 30 ml distilled water and heated to 70° C. to dissolve the complex. The solution was then cooled to RT. Thereafter, aqueous solution of silver nitrate ((135.9 mg, 0.8 mmoles) in 10 ml of water) was added drop-wise to the solution of starting material under stirring (400 rpm). The formation of translucent white precipitate of silver chloride was commenced immediately after the addition of the aqueous solution. The mixed solution was stirred for 3 hr at RT under light-shielding conditions. The resulting precipitate of silver chloride was filtered off (Corning polystyrene Filter System, Corning, Amsterdam, Netherlands) (0.22 μM) and washed with water. The combined filtrate was used in the following step without further treatment.

The Pt (II) monostearate complex was prepared as follows. To an aqueous solution of sodium stearate ((245.15 mg, 0.8 mmoles) (Sigma) in 10 ml of water) was added the aqueous solution obtained above and stirred at RT for 3 wk under light-shielding conditions to complete the reaction. The translucent white precipitate formed was filtered off, washed with a small amount of ether, and dried in vacuum desiccator to obtain the crude product.

Because the crude product contained a mixture of mono- and di-fatty acid Pt derivatives, and the mono-fatty acid Pt derivative was soluble in chloroform, chloroform was used to purify the mixture. The crude product was suspended in 25 ml chloroform in conical tube, vortex mixed for 5 min, and kept at RT for 24 hr. Tubes were then centrifuged (5000 rpm, 10 min) (Beckman-Coulter, Inc.), and the supernatant was carefully transferred into glass vials and vacuum dried to obtain the Pt (II) monostearic acid complex (yield=6.08%).

### Example 3

### Synthesis of cis-Diamine Pt (II) Chloride Monomyristic Acid (Pt-MMA)

The cisplatin intermediate was prepared as follows. Cis-dichlorodiamine Pt (II) (Sigma, Louis, Mo.) (240 mg, 0.8 mmoles) was suspended in 30 ml distilled water and heated to 70° C. to dissolve the complex. The solution was then cooled to RT. Thereafter, an aqueous solution of silver nitrate (Sigma) (135.9 mg, 0.8 mmoles) in 10 ml of water was added drop-wise to the solution of starting material under stirring (400 rpm). The formation of translucent white precipitate of silver chloride was commenced immediately after the addition of the aqueous solution. The mixed solution was stirred for 3 hr at RT under light-shielding conditions. The resulting precipitate of silver chloride was filtered off (Corning polystyrene Filter System, Corning) (0.22 μM) and washed with water. The combined filtrate was used in the following step without further treatment.

Sodium myristate (Sigma) (200 mg, 0.8 mmoles) in 10 ml of water was added to the aqueous solution obtained above and stirred at RT for 3 wk under light-shielding condition to complete the reaction there between.

The translucent white precipitate formed was filtered off, washed with a small amount of ether, and dried in a vacuum desiccator to obtain the crude product. The crude product consisted of a mixture of mono- and di-fatty acid Pt derivatives. Because the mono-fatty acid Pt derivative was soluble in chloroform, chloroform was used to purify the mixture. The crude product was suspended in 25 ml chloroform in conical tube, vortex mixed for 5 min, and kept at RT for 24 hr. Tubes were then centrifuged (5000 rpm, 10 min), and the supernatant was transferred into glass vials and vacuum dried to obtain the dry Pt (II) mono-myristic acid complex (yield=24.8%).

### Example 4

### Synthesis of Diaminocyclohexane Platinum-3,5 Diiodosalicylate (Pt-SA)

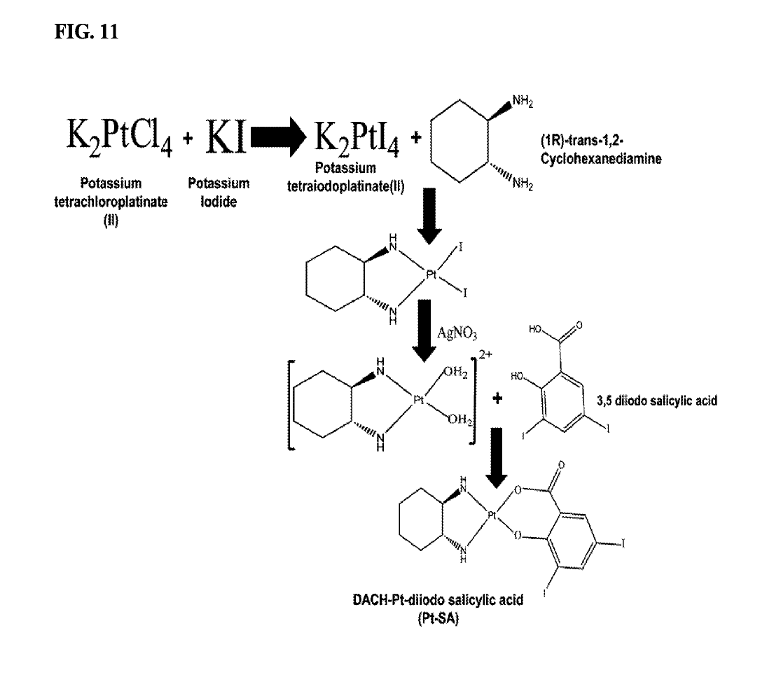

The DACH-Pt intermediate was prepared as shown in FIG. 11. Potassium tetrachloroplatinate (II) (Sigma) (208 mg, 50 mM) was dissolved in distilled water, to which potassium iodide (Sigma) (830 mg, 0.5 mM) dissolved in 1 ml of distilled water was added and stirred for 5 min at RT. (1R, 2R)-trans-1,2-cyclohexanediamine (DACH) (Sigma) (57 mg, 50 mM) was then added to the above solution and stirred for 1 hr. The resulting precipitate was filtered off using Whatman paper and washed with water, ethanol, and acetone. The sample was Vacuum dried to obtain the dry product of DACH-Pt (235 mg). This product was suspended in 200 ml of distilled water and added with silver nitrate (Sigma) (115 mg, 3.37 mM) and stirred at RT for 24 hr. The resulting precipitates of silver chloride were filtered off with Corning polystyrene Filter System (Corning) (0.22 μM) and washed. The combined filtrate was used in the following step without further treatment.

The Pt-SA complex was prepared as follows. 3,5 diiododisodium salicylic acid (DISA) (Sigma) (182 mg, 2.10 mM) was added to the above filtrate and stirred for 1 hr. The resulting Pt-SA precipitate was filtered off using Whatman paper and washed with water. The final product was dried under a vacuum.

### Example 5

### Synthesis of EGFR_{BP}-PEG-DSPE

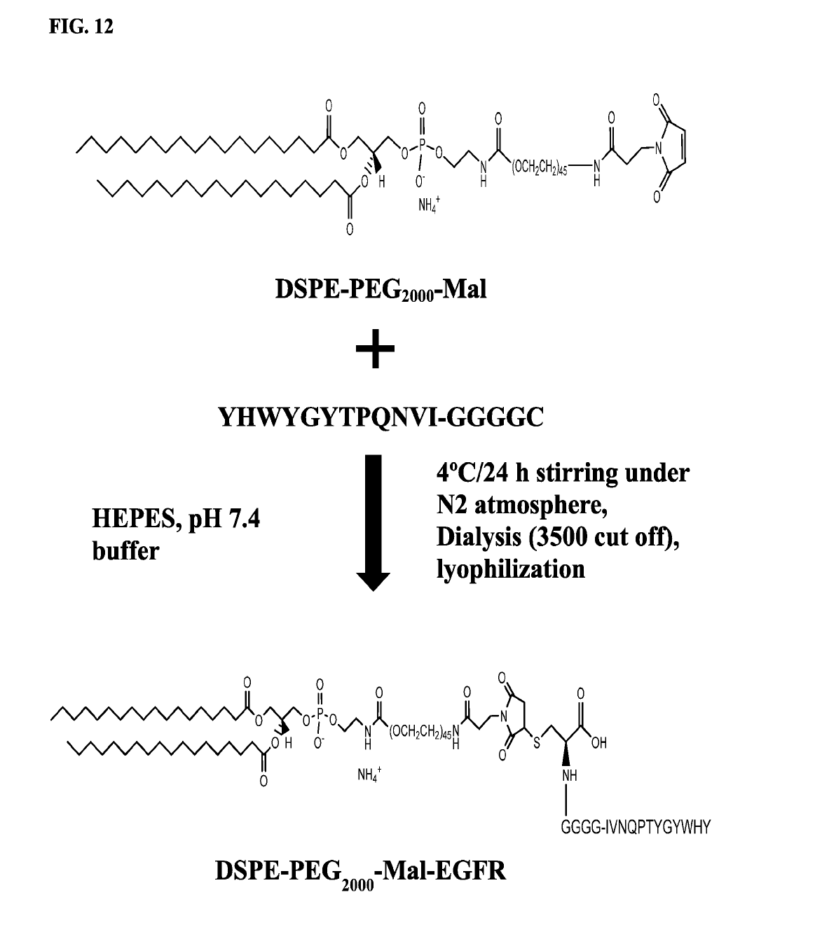

EGFR_{BP}-PEG-DSPE was prepared according to the scheme shown in FIG. 12.

Briefly, the synthetic EGFR-targeting peptide Y-H-W-Y-G-Y-T-P-Q-N-V-I (SEQ ID NO:1) with a linker sequence G-G-G-G-C(SEQ ID NO:2) was synthesized by standard peptide organic synthesis methods. The carboxyl group of terminal cysteine of the peptide was reacted with the maleimide of the PEG₂₀₀₀-DSPE construct. To accomplish this reaction, 9.4 mg of EGFR-binding peptide Y—H-W YGYTPQNVI G-G-G-G-C(SEQ ID NO:3) was added to 14.7 mg MAL-PEG₂₀₀₀-DSPE (2942 kd mol. wt.) dissolved in HEPES (4-(2-hydroxyethyl)-1-piperazineethanesulfonic acid) buffer (pH 7.4) solution at 1:1 molar ratio while mixing at 400 rpm) under nitrogen at 4° C. for 24 hr.

The EGFR_{BP} conjugate was then purified by dialysis against deionized distilled water at RT using a 3500 molecular weight cut-off membrane (Spectrapore, Spectrum Laboratories, Rancho Dominguez, Calif.). The purified sample was then transferred into tubes and freeze-dried for 24 hr. The sample was stored at −20° C. until use.

EGFR_{BP}-PEG₂₀₀₀DSPE conjugate formation was confirmed by nuclear magnetic resonance spectroscopy (NMR) analysis. A 2 mg sample was dissolved in 1 ml DMSO (dimethyl sulfoxide) and the NMR spectra was recorded using a Varian 400AS Spectrometer (400 MHz, Varian Inc., Palo Alto, Calif.).

### Example 6

### Synthesis of Folate-Targeting Ligand DSPE-PEG-Cys-FA

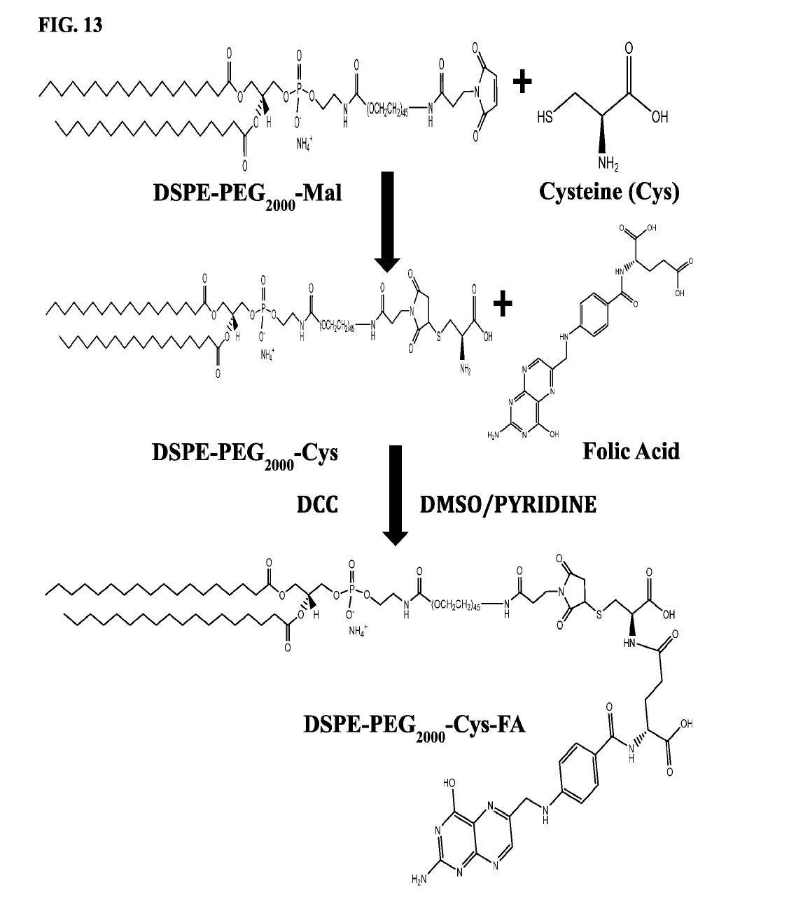

The DSPE-PEG-Mal complex was prepared according to the scheme in FIG. 13. DSPE-PEG-Mal (100 mg, 1.3596 mM) was added to cysteine (8.24 mg, 2.72 mM) in a 1:2 molar ratio in HEPES buffer (25 ml) and the coupling reaction was carried out overnight at 4° C. under a nitrogen environment. The next day, excess cysteine was dialyzed out for 24 hr using 2000 Da cut-off dialysis bags. The outside water was changed every 2 hr to facilitate dialysis.

A purified sample was freeze-dried and characterized by NMR. 51 mg of DSPE-PEG-Cys was dissolved in 6 ml dry DMSO containing 13 mg folic acid. 3 ml pyridine was added to the solution followed by 16 mg of N,N′-dicyclohexylcarbodiimide. The coupling was carried out for 4 hr at RT with continuous mixing. The sample was dialyzed in water using 2000 Da cut-off dialysis bags. Outside water was changed every 2 hr for 24 hr to facilitate dialysis. Purified sample was freeze-dried and characterized by NMR.

### Example 7

### Synthesis of Gd⁺³-DTPA-PE

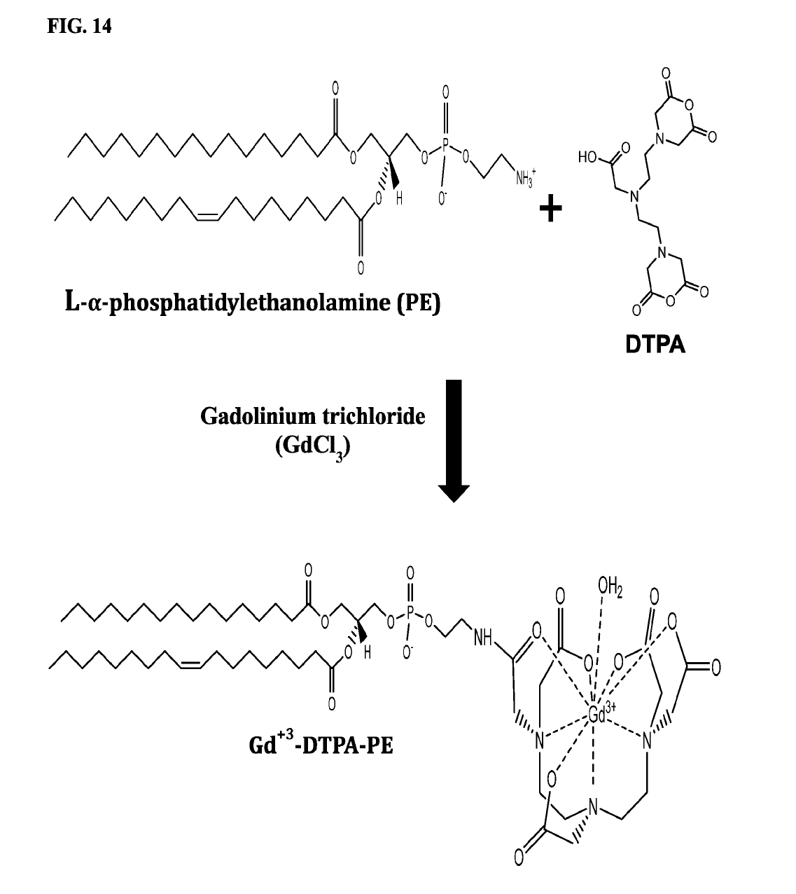

Gd⁺³-DTPA-PE chelate was prepared according to the scheme shown in FIG. 14. 30 μl of triethylamine (Sigma) was added to 100 mg of L-a-phosphatidylethanolamine, transphosphatidylated (egg chicken) (841118C, Avanti Polar Lipids, Birmingham, Ala.) dissolved in 4 ml of chloroform (extra dried). This solution was then added drop-wise to 400 mg (1 mM) of diethylene triaminepentacetic dianhydride (DTPA anhydride) (Sigma) in 20 ml of dimethylsulfoxide and the mixture was stirred for 3 hr under nitrogen atmosphere at RT. Nitrogen was then blown on to a sample to remove the chloroform.

The DTPA-PE conjugate was then purified by dialysis against deionized distilled water at RT using a 3000 molecular weight cut-off membrane (Spectrapore, Spectrum Laboratories). The purified sample was then transferred into tubes and freeze-dried for 48 hr. The DTPA-PE complex formation and purity of the complex were monitored by thin layer chromatography (TLC) using a mobile phase of chloroform:methanol:water at a 3.25:1.25:0.5 (v/v) ratio and using ninhydrin as a visualizing reagent. For this, reactants (DTPA, PE) and complex (DTPA-PE) were dissolved in chloroform and placed on a TLC plate and developed in the mobile phase. Ninhydrin solution was then sprayed and the spots and their retention times were compared for the formation of the complex.

18.5 mg (10.0 mM) of gadolinium (III) chloride hexahydrate (Sigma) in 0.1 ml of water was then added drop-wise to the 100 mg of DTPA-PE complex dissolved in 20 ml of DMSO and the reaction mixture was stirred (400 rpm) for 1 hr.

An Arsenazo III assay was used to monitor the reaction and formation of the Gd-DTPA-PE complex. 10 μl of reaction mixture was added to 0.2 mM of Arsenazo III (Pointe Scientific) in water and observed for the color change (pink to blue). No change in solution color indicated that Gd conjugated to DTPA-PE (free Gd turns Arsenazo III solution to a blue color).

The resulting Ge-DTPA-PE conjugate was purified by dialysis against deionized distilled water at RT using a 3000 molecular weight cut-off membrane (Spectrapore, Spectrum Laboratories). The purified sample was then transferred into tubes and freeze-dried for 48 hr. The conjugate was stored at −20° C. until use.

### Example 8

### Preparation of a Pt-MMA Nanoemulsion Formulation

The oil phase of this oil-in-water nanoemulsion was prepared as follows. 20.8 mg of Pt monomyristate (Pt-MMA) was dissolved in chloroform (extra dry) in a glass scintillation vial. 20.8 mg of Pt-MMA is equivalent to 5 mg of cisplatin based on Inductively Coupled Plasma Mass Spectrometry (ICP-MS) analysis. ICP-MS is a type of mass spectrometry, which is capable of detecting metals and several non-metals by ionizing the sample with inductively coupled plasma and then using a mass spectrometer to separate and quantify those ions. Flax seed oil (1 g) was placed in a scintillation glass vial. Pt-MMA solution was added and nitrogen gas was blown on the sample to evaporate chloroform and to form the oil phase.

The aqueous phase of this oil-in-water nanoemulsion was prepared as follows. 120 mg egg lecithin (Lipoid E 80, Lipoid GMBH, Ludwigshafen, Germany), 15 mg PEG₂₀₀₀DSPE (Genzyme, Cambridge, Mass.) was added to 4 ml of 2.21% w/v glycerol (Sigma) solution in a glass scintillation vial made in water for injection. The mixture was stirred (400 rpm) for 1 hr to achieve complete dissolution of these excipients.

The aqueous and oil phases from above steps were heated to 60° C. for 2 min in a water bath, and the aqueous phase was added to the oil phase, and vortex mixed for 1 min. The resulting mixture was passed through a LV1 Microfluidizer (Microfluidics Corp., Newton, Mass.) at 25,000 psi for 10 cycles. Product entered the Microfluidizer system via the inlet reservoir and was powered by a high-pressure pump into the interaction chamber at speeds up to 400 m/s. It was then effectively cooled and collected in the output reservoir.

These steps resulted in the production of a stable cis-diamine Pt (II) chloride monomyristate acid (Pt-MMA) nanoemulsion formulation or nanoemulsion.

### Example 9

### Preparation of cis-Diamine Pt (II) Chloride Monopalmitic Acid Pt-MPA Nanoemulsion

The oil phase of this oil-in-water nanoemulsion was prepared as follows. Pt monopalmitic (Pt-MPA) (20.8 mg, equivalent to 5 mg of cisplatin based on ICP-MS analysis) was dissolved in chloroform (extra dry) in a glass scintillation vial. Flax seed oil (1 g) was weighted into a scintillation glass vial to which the Pt-MPA solution was added. Nitrogen gas was then blown on the sample to evaporate chloroform and to form the oil phase.

The aqueous phase of this oil-in-water nanoemulsion was prepared as follows. 120 mg egg lecithin (Lipoid E 80, Lipoid GMBH) and 15 mg PEG2000DSPE (Genzyme) were added to 4 ml of 2.21% w/v glycerol (Sigma) solution in a glass scintillation vial made in water for injection. The mixture was stirred (400 rpm, Corning Stirrer plate) for 1 hr to achieve complete dissolution of these excipients.

The aqueous and oil phases from above steps were heated to 60° C. for 2 min in a water bath, and then the aqueous phase was added to the oil phase, and vortex mixed for 1 min. The resulting mixture was passed through a LV1 Microfluidizer (Microfluidics Corp.) at 25,000 psi for 10 cycles, resulting in the production of a stable cis-diamine Pt (II) chloride monopalmitic acid Pt-MPA nanoemulsion.

### Example 10

### Preparation of cis-Diamine Pt (II) Chloride Monostearic Acid (Pt-MSA) Nanoemulsion

The oil phase of this oil-in-water nanoemulsion was prepared as follows. Pt monomyristate (Pt-MSA) (20.8 mg, equivalent to 5 mg of cisplatin based on ICP-MS analysis) was dissolved in chloroform (extra dry) in a glass scintillation vial. Flax seed oil (1 g) was weighted into a scintillation glass vial. Pt-MSA solution was added to flax seed oil and nitrogen gas was blown on the sample to evaporate chloroform and to form the oil phase.

The aqueous phase of this oil-in-water nanoemulsion was prepared as follows. 120 mg egg lecithin (Lipoid E 80, Lipoid GMBH), 15 mg PEG2000DSPE (Genzyme) were added to 4 ml of 2.21% w/v glycerol (Sigma) solution in a glass scintillation vial made in water for injection. The mixture was stirred (400 rpm) for 1 hr to achieve complete dissolution of these excipients.

The aqueous and oil phases from above steps were heated to 60° C. for 2 min in a water bath, and then the aqueous phase was added to the oil phase, and vortex mixed for 1 min. The resulting mixture was passed through a LV1 Microfluidizer (Microfluidics Corp.) at 25,000 psi for 10 cycles, resulting in the production of a stable cis-diamine Pt (II) chloride monostearate acid (Pt-MSA) nanoemulsion.

### Example 11

### Preparation of Diaminocyclohexane Platinum-3,5 Diiodosalicylate (Pt-SA) Nanoemulsion

The oil phase of this oil-in-water nanoemulsion was prepared as follows. Diaminocyclohexane (DACH) platinum-3,5 diiodosalicylate (Pt-SA) (10 mg) was dissolved in chloroform (extra dry) in a glass scintillation vial. Flax seed oil (1 g) was weighted into scintillation glass vial. Pt-SA solution is added to flax seed oil and the nitrogen gas was blown on the sample to evaporate chloroform and to form the oil phase.

The aqueous phase of this oil-in-water nanoemulsion was prepared as follows. 120 mg egg lecithin (Lipoid E 80, Lipoid GMBH), 15 mg PEG₂₀₀₀DSPE (Genzyme) were added to 4 ml of 2.21% w/v glycerol (Sigma) solution in glass scintillation vial made in water for injection. The mixture was stirred (400 rpm, Corning Stirrer plate) for 1 hr to achieve complete dissolution of these excipients.

The aqueous and oil phases from above steps were heated to 60° C. for 2 min on water bath and then aqueous phase was added to the oil phase, and vortexed for 1 min. The resulting mixture was passed through LV1 Microfluidizer (Microfluidics Corp.) at 25,000 psi for 10 cycles, resulting in the production of a Pt-SA formulation with the droplet size below 150 nm.

### Example 12

### Cellular Uptake Studies

The following assay demonstrates the effect that targeted nanoemulsion formulations have on cellular uptake. Uptake was measured in ovarian SKOV3 cells using fluorescence. SKOV3 cells growing on cover slips in 6-well plate at 3000 cells/well were incubated with the fluorescently labeled nanoemulsion formulations for 5 min, 15 min, and 30 min. At the end of incubation period, cells were washed thrice with phosphate buffered saline (PBS) and incubated with Lyso Tracker and DAPI for 10 min, which stains lysosomes and nucleus of the cells, respectively. Cells were further washed with PBS, inverted and mounted on glass slides using Flouromount G mounting media.

DIC/Fluorescent images of fluorescently labeled SKOV3 cells treated with nanoemulsion formulation according to the present disclosure were acquired using a Confocal Zeiss LSM 700 microscope with an object 63× oil immersion over a 30 min period.

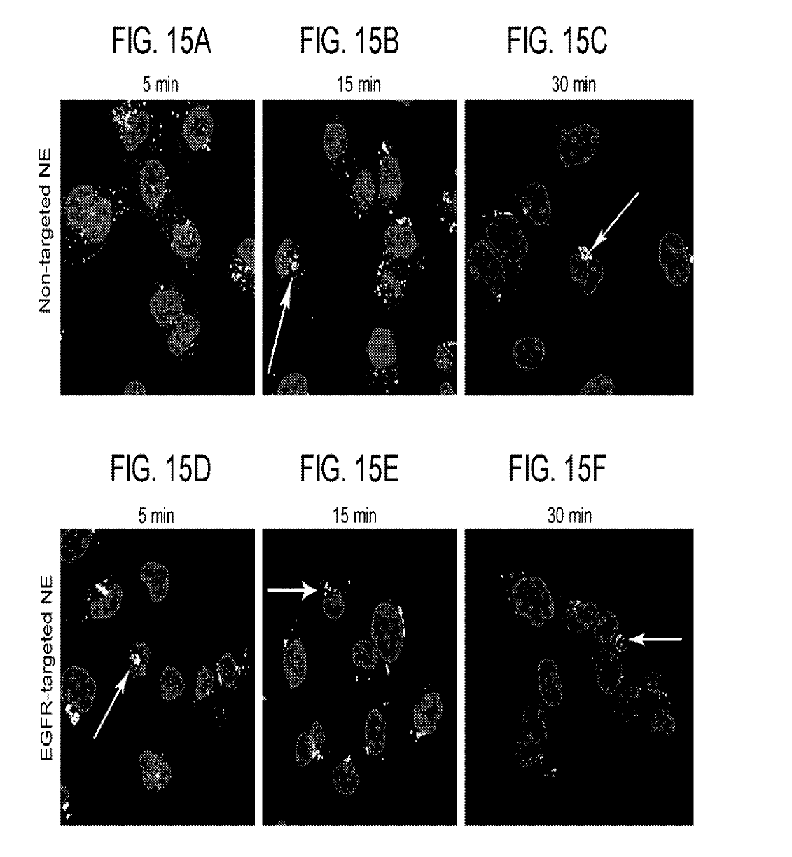

FIGS. 15A-15F display the cellular uptake of non-targeted nanoemulsion formulations (FIGS. 15A-15C) and EGFR-targeted nanoemulsion formulations (FIGS. 15D-15F). 0.01% NBD-Ceramide at 0.01% w/v was incorporated in all formulations. 0.02% EGFR was incorporated into the targeted formulation. Lyso Tracker and DAPI were used to monitor the co-localization of the nanoemulsion formulations in the SKOV3 cells.

These images show that NBD-CER (arrows) co-localized with Lyso Tracker, indicating the entry of NBD-CER into lysosomes. The study demonstrates that the nanoemulsion formulations of the present disclosure are able to evade the drug degradative lysosomal pathway, thereby enhancing drug concentrations in cells in vitro.

### Example 13

### Efficacy of Nanoemulsion Formulations

In order to determine if the nanoemulsion formulations according to the disclosure produce a cytotoxic effect, the following experiments were done on SKOV3 cells.

A tetrazolium (MTT) assay was performed, which measures the activity of cellular enzymes that reduces the MTT dye to insoluble formazan. SKOV3 cells were treated with corresponding test nanoemulsions or control solutions as indicated below in Table VI. Polyethylenimine at 50 μg/ml was used as a positive control for cytotoxicity. The effect of cis-Pt in solution, nanoemulsion formulations, and EGFR-targeted nanoemulsion formulations of the disclosure on the viability of ovarian SKOV3 cells were studied and measured after 72 hr treatment. After the completion of treatment, cells were incubated with MTT reagent (50 μg/well) for 2 hr. The resulting formazan crystals were dissolved in dimethyl sulfoxide (150 μg/well) and measured at 570 nm in the Plate reader (Synergy HT, Biotek Instruments, Winooski, Vt.).

The concentration of drug that inhibits fifty percent of growth is known as the 50% growth inhibitory concentration (IC₅₀). Using the dose response curves (not shown), the IC₅₀ values were calculated and are shown in Table VI. Values are shown as mean±SD, n=8. All IC₅₀ values were obtained by analyzing the MTT assays results using Graphpad Prism 5 scientific data analysis software.

TABLE VI
Inhibition of Growth of SKOV-3 Ovarian Cancer Cells
Inhibitory Concentration Analysis

| Treatment | IC₅₀ (nM) |
|---|---|
| Cisplatin in Solution | 18,7000 ± 110 |
| Pt-MMA Nanoemulsion | 19,500 ± 100 |
| Pt-MMA Targeted Nanoemulsion | 2,400 ± 110 |
| Pt-MPA Nanoemulsion | 13,000 ± 130 |
| Pt-MPA Targeted Nanoemulsion | 4,300 ± 100 |
| Pt-MSA nanoemulsion | 529 |
| CER in Solution | 10,000 ± 100 |
| CER Nanoemulsion | 9,190 ± 120 |
| CER Targeted Nanoemulsion | 8,300 ± 110 |
| (Pt-MMA + CER) Nanoemulsion | 460 ± 10 |
| (Pt-MMA + CER) Targeted Nanoemulsion | 360 ± 10 |
| (Pt-MPA + CER) nanoemulsion | 600 ± 20 |
| (Pt-MPA + CER)-targeted nanoemulsion | 480 ± 10 |

The optimum concentration of multiple chemotherapeutic agents combined in the nanoemulsion formulations of the present disclosure can be determined by calculating the combination index from the dose response curves of the single agents (Chou (2006) Pharmacol. Rev. 58(3): 621-681). This method uses the isobologram equation below to determine combination index (CI):

CI=(a/A)+(b/B)

where, “a” is the primary therapeutic IC₅₀ in combination with secondary therapeutic at concentration “b.” “A” is the primary therapeutic IC₅₀ without secondary therapeutic; and “B” is the secondary therapeutic IC₅₀ in the absence of primary therapeutic. The CI represents the degree of interaction between two drugs regardless of mechanism. A CI value lower than 1.0 indicates synergy, while a CI value greater than 1.0 indicates that the drugs are antagonists. If drugs are synergistic the relative dose needed to get the same effect is reduced and is known as the “dose reduction index” (DRI). DRI is a measure of decrease in drug concentration for a synergistic combination as compared with the concentration of each drug alone.

For the nanoemulsion formulations of the present disclosure, the ratio of Pt:CER was determined in order to identify ratios that could reduce the IC₅₀ of Pt. Table VII shows CI and DRI for the combination of ceramide with Pt-MMA or Pt-MPA.

TABLE VII
Pt and CER Combination Index and Dose Reduction Index Analysis

| | | Dose | Dose | |
|---|---|---|---|---|
| | | Reduction | reduction | Degree of |
| Combination | Combination | Index | Index (DRI) | Combination |
| Treatment | Index (CI) | (DRI) [Pt] | [CER] | Effect |
| Pt-MMA + | 0.3326 | 11.38 | 4.09 | Synergism |
| CER NE | | | | |
| Pt-MPA + CER | 0.5746 | 17.45 | 1.93 | Synergism |
| NE | | | | |

The data in Table VII demonstrate that both MMA-Pt and MPA-Pt act synergistically with CER in killing cancer cells. Of the Pt(II) complex:CER ratios calculated, 1:5 was determined to be optimal for in vitro cytotoxicity and for the formation of stable Pt/CER nanoemulsion formulations.

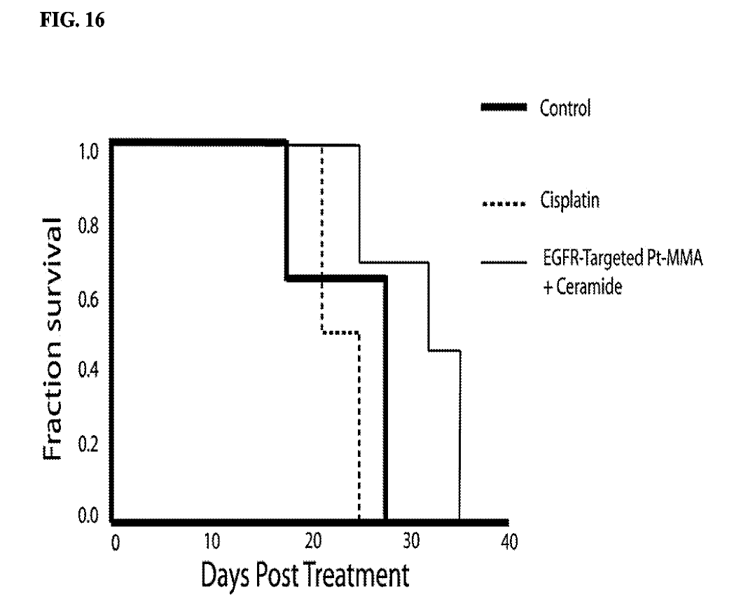

In order to determine if the nanoemulsion formulations according to the disclosure produce therapeutic efficacy against cancer in vivo, the following experiment was performed. Orthotropic tumors were developed in 30 Nu/Nu female mice, each weighing approximately 20 g (Charles River Laboratories, Cambridge, Mass.). The mice were injected intraperitoneally (ip) with 4×10⁶ SKOV3 human ovary cancer cells (ATCC, Manassas, Va.) suspended in phosphate buffered saline100 μl PBS. The 30 mice were divided into 3 test groups of 10 individual mice. Each mouse was then dosed every 7 d for 5 wk with either nothing (control group) or 1 of 2 test compounds. The first test compound (cisplatin) was administered as a comparative sample in 5 mg/kg doses. The second test compound (EGFR-targeted nanoemulsion formulation containing Pt-MMA and ceramide in a 1:5 molar ratio) was administered in 3 cycles of 5 mg/kg platinum and an additional 2 cycles of 7.5 mg/kg platinum. The doses of Pt-MMA (21 mg/kg) and cis-Platin (5 mg/kg) were calculated so that animals received equivalent amounts of Pt. The survival time of each group of mice was determined and the median survival time (days) calculated using Graphpad Kaplan-Meyers survival analysis software on the basis of the observed survival time of each mouse. The results are shown in Table VIII and FIG. 16.

TABLE VIII
Therapeutic Efficacy of Mono-Fatty Acid Complex

| | | | Average | Time for | | Median Survival |
|---|---|---|---|---|---|---|
| | | Average | Tumor | First Tumor | | Significance |
| | | Tumor | Size at 21 | to Reach | Median | Compared to |
| | Dose | Starting Size | Days | 1000 mm³ | Survival | Control (P- |
| Treatment | (mg/kg) | (mm³) | (mm³) | (days) | (days) | Value) |
| Control | — | 166 ± 47 | 1071 ± 428 | 11 | 23 | — |
| Cisplatin | 5 | 171 ± 43 | 640 ± 314 | 17 | 28 | 0.0606 |
| EGFR-T Pt- | 21 | 167 ± 49 | 688 ± 288 | 21 | 32 | 0.0044 |
| MMA CER | | | | | | |
| NE | | | | | | |

These results show that the fractional survival for groups treated with the EGFR-targeted nanoemulsion formulation of the present disclosure significantly improved median survival as compared to that of the control and free cisplatin group. Encapsulation of Pt-MMA and ceramide in the targeted nanoemulsion formulation sequesters them from normal tissue to reduce therapy related systemic toxicity, while still allowing the Pt-MMA and ceramide combination to inhibit the division of cancer cells in tumors. The nanoemulsion formulations of the present disclosure, in which targeting ligands are present, are thus useful as anticancer delivery systems. These nanoemulsion formulations allow for a more efficient chemotherapeutic delivery system, which had reduced systemic toxicity while functioning to inhibit the division of cancer cells.

Encapsulation of Pt-MMA and ceramide as part of the oil core of the nanoemulsion formulation according to the disclosure and inclusion of EGFR-targeting modified lipids allowed for targeting moieties to be attached to an amphiphile of the interfacial surface membrane of the nanoemulsion formulation.

### Example 14

### In Vivo Pharmacokinetic Studies of Nanoemulsion Formulations

The following study was conducted to determine the pharmacokinetics (PK) of nanoemulsion formulations of the present disclosure, which were Gd-labeled and contain a Pt(II) complex.

EGFR-targeted, Gd-labeled Pt-MMA or non-targeted, Gd-labeled Pt-MMA were administered at 21 mg/kg of Pt and 0.072 mM/kg of Gd to non-tumor bearing female Nu/Nu mice via an iv route of administration. Blood samples were collected over a 24 hr period and processed for Pt and Gd using a validated ICP-MS method.

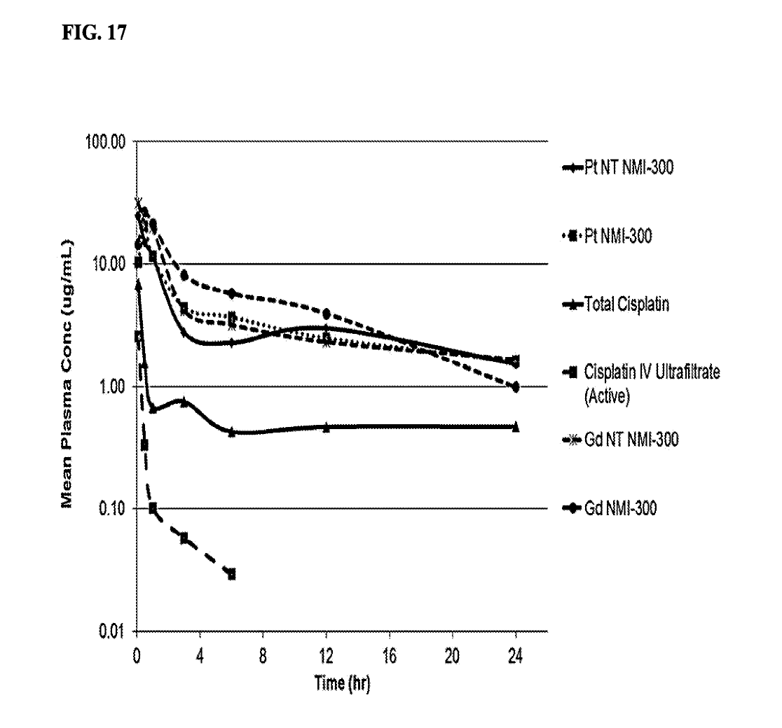

FIG. 17 shows that levels of plasma Pt PK are markedly improved compared to that of cisplatin. The Gd and Pt concentrations versus time profiles closely tracked each other, showing that the nanoemulsions remain intact and functional for an extended period of time.

Table IX shows non-compartmental pharmacokinetic profile of non-targeted (NT) vs targeted (T) nanoemulsion formulations (Gd (0.072 mmol/kg or 11.322 mg/kg) and Pt (5 mg/kg)) following iv administration. This table highlights the enhanced PK profile Pt/Gd delivered by and administered iv. Pharmacokinetic parameters were calculated by non-compartmental analysis using Phoenix WinNonlin 6.2 version. Lp. studies showed similar results with blood concentrations peaking at 3 hr and ending at nearly identical values to i.v. treated mice for both Pt and Gd. All parameters suggest the both NMI-300 & NMI-300(NT) are long circulating and remain intact. There is a significant increase in half life of the Pt compared to published values for cisplatin (5.1 hr) & carboplatin (0.84 hr).

TABLE IX

| | | Pt-MMA | | Pt-MMA | |
|---|---|---|---|---|---|
| | | NE NT | | NE T | |
| | Sample | [Gd] | [Pt] | [Gd] | [Pt] |
| HL | hr | 9 | 12 | 6 | 10 |
| Tmax | hr | 0.08 | 0.08 | 0.5 | 0.5 |
| Cmax | ug/mL | 32 | 25 | 27 | 20 |
| AUClast | hr * ug/mL | 99 | 108 | 131 | 109 |
| AUCINF | hr * ug/mL | 120 | 82 | 139 | 86 |
| Vz | mL/kg | 1185 | 804 | 696 | 657 |
| Cl | mL/hr/kg | 94 | 46 | 81 | 46 |
| AUMClast | hr * hr * ug/mL | 635 | 643 | 819 | 651 |
| AUMCINF | hr * hr * ug/mL | 1392 | 1747 | 1095 | 1536 |
| MRTlast | hr | 6 | 8 | 6 | 8 |
| MRTINF | hr | 12 | 16 | 8 | 14 |
| Vss | mL/kg | 1096 | 742 | 638 | 643 |

### Example 15

### In Vivo MRI Studies Using Gd-Labeled Nanoemulsion Formulations

That a Gd-based MRI contrasting agent is useful in the nanoemulsion formulation according to the disclosure was demonstrated as follows:

Three female Nu/Nu mice (Charles River Laboratories, Cambridge, Mass.) each weighing approximately 20 g and bearing subcutaneous SKOV3 tumors (human ovary cancer cells, American Type Culture Collection (ATCC, Manassas, Va.) approximately 200 mm to 300 mm in size, were used as test subjects. The first mouse was intravenously injected with gadopentetic acid containing a 0.072 mmol/Kg dose of the gadolinium-based MRI contrasting agent Gd-DTPA-PE (Magnevist™). The second mouse was intravenously injected with a non-targeted Gd-labeled nanoemulsion formulation of the present disclosure containing a 0.072 mmol/Kg dose of the gadolinium-based MRI contrasting agent Gd-DTPA-PE. The third mouse was intravenously injected with an EGFR-targeted nanoemulsion formulation of the present disclosure containing a 0.072 mmol/Kg dose of the gadolinium-based MRI contrasting agent Gd-DTPA-PE. All three mice were full body scanned and imaged using a Bruker Biospec 20/70 MRI machine over a period of 24 hr.

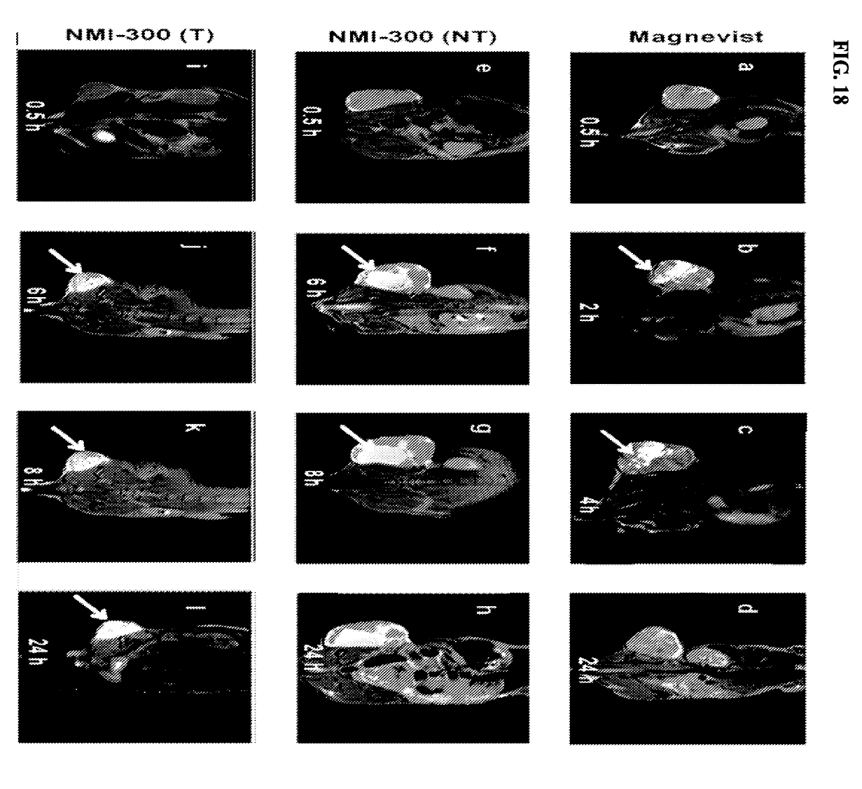

Representations of the resulting MRIs of a mouse injected with Magnevist™ (Magnevist™), a mouse injected with the non-targeted nanoemulsion formulation (Non-Targeted), and a mouse injected with the EGFR-targeted nanoemulsion formulation (Targeted) can be seen in FIG. 18. These images show preferential accumulation of the Gd-containing nanoemulsion formulations of the present disclosure in the subcutaneous flank SKOV3 tumors compared to that of the contrasting agent contron Magnevist™. The Magnevist™ control was observed to show contrast enhancement of tumors between 2 hr to 4 hr; whereas the Gd-labeled targeted and non-targeted nanoemulsion formulations of the present disclosure shows contrast enhancement of tumors between 6 hr to 24 hr. The Magnevist™ control rapidly accumulated in the tumor over the first hour, but then cleared and resolved to near baseline by the 6th hr. In contrast, the nanoemulsion formulations of the present disclosure enhanced tumor imaging accumulated and remained in the tumor over a longer period of time, thereby enhancing tumor imaging.

These studies show that the Gd-containing nanoemulsion formulations of the present disclosure are useful MRI agents, and are effective at imaging tumors over a longer period of time than the pure imaging agent Magnevist™. Additionally, these tumor images can be used to measure the accumulation of Pt complex nanoemulsion formulation in the tumor.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

What is claimed is:

1. A nanoemulsion formulation comprising:
an oil phase;
an interfacial surface membrane;
an aqueous phase; and
a chemotherapeutic agent comprising a hydrophobic platinum derivative which is not carboplatin, or cisplatin, the chemotherapeutic agent being dispersed in the oil phase.

2. The nanoemulsion formulation of claim 1, wherein the oil phase comprises flaxseed oil, omega-3 polyunsaturated fish oil, omega-6 polyunsaturated fish oil, safflower oil, olive oil, pine nut oil, cherry kernel oil, soybean oil, pumpkin oil, pomegranate oil, primrose oil, or combination thereof.

3. The nanoemulsion formulation of claim 1, wherein the interfacial surface membrane phase comprises an emulsifier and/or a stabilizer.

4. The nanoemulsion formulation of claim 3, wherein the emulsifier comprises egg lecithin, egg phosphatidyl choline, soy lecithin, phosphatidyl ethanolamine, phosphatidyl inositol, dimyristoylphosphatidyl choline, dimyristoylphosphatidyl ethyl-N-dimethyl propyl ammonium hydroxide, hydrogenated soy phosphatidylcholine, 1,2-distearoyl-sn-glycero-3-phosphocholine, 1-palmitoyl-2-oleoyl-sn-glycero-3-phosphocholine, or combination thereof.

5. The nanoemulsion formulation of claim 3, wherein the stabilizer comprises a polyethylene glycol derivative, a phosphatide, a polyglycerol mono oleate, or a combination thereof.

6. The nanoemulsion formulation of claim 5, wherein the polyethylene glycol derivative is PEG₂₀₀₀DSPE, PEG₃₄₀₀DSPE, PEG₅₀₀₀DSPE, or combination thereof.

7. The nanoemulsion of claim 5, wherein the polyethylene glycol in the polyethylene glycol derivative has a molecular weight of from 1 kD to 20 kD, from 5 kD to 20 kD, or from 10 kD to 20 kD.

8.-9. (canceled)

10. The nanoemulsion formulation of claim 1, wherein the hydrophobic platinum derivative comprises diaminocyclohexane (DACH) platinum-3,5 diiodosalicylate (Pt-SA).

11. The nanoemulsion formulation of claim 1, further comprising a chemopotentiator.

12. The nanoemulsion formulation of claim 11, wherein the chemopotentiator comprises ceramide or a derivative thereof.

13. The nanoemulsion formulation of claim 12, wherein the chemopotentiator comprises C6-ceramide.

14. The nanoemulsion formulation of claim 1, further comprising C6-ceramide.

15.-20. (canceled)

21. The nanoemulsion formulation of claim 1, further comprising a targeting ligand.

22. The nanoemulsion formulation of claim 21, wherein the targeting ligand comprises an EGFR-targeting ligand, a folate receptor-targeting ligand, or a combination thereof.

23. The nanoemulsion formulation of claim 22, wherein the targeting ligand is an EGFR-targeting ligand comprising peptide 4, an anti-EGFR immunoglobulin or EGFR-binding fragment thereof, EGa1-PEG, or combination thereof.

24. The nanoemulsion formulation of claim 23, wherein the targeting ligand is a folate-targeting ligand comprising DSPE-PEG-cysteine-folic acid, DSPE-PEG₂₀₀₀ folate, DSPE-PEG₃₄₀₀ folate, DSPE-PEG₅₀₀₀ folate, an anti-folate receptor immunoglobulin or folate receptor-binding fragment thereof, or combination thereof.

25. The nanoemulsion formulation of claim 23, wherein the PEG in the targeting ligand EGa1-PEG has a molecular weight of from 1 kD to 20 kD, from 5 kD to 20 kD, or from 10 kD to 20 kD.

26. The nanoemulsion formulation of claim 1, further comprising an imaging agent.

27. The nanoemulsion formulation of claim 26, wherein the imaging agent is an MRI contrasting moiety.

28. The nanoemulsion formulation of claim 27, wherein the MM contrasting moiety comprises gadolinium, iron oxide, iron platinum, manganese, or a combination thereof.

29. The nanoemulsion formulation of claim 28, wherein the MM contrasting moiety comprises Gd-DTPA-PE, Gd-DOTA-PE, Gd-PAP-DOTA, or combination thereof.

30. A method of imaging a cancer in a patient, comprising administering to a patient having the cancer an amount of the nanoemulsion formulation of claim 26 sufficient to image the cancer.

31. A method of inhibiting the growth of, or killing, a cancer cell, comprising contacting the cancer cell with an amount of the nanoemulsion formulation of claim 1 that is toxic to, or which inhibits the growth of, or kills, the cancer cell.

32. The method of claim 31, wherein the cancer cell is in a mammal, and the contacting step comprises administering to the mammal a therapeutically effective amount of the nanoemulsion formulation.


SEQUENCE LISTING (3 sequences)

SEQ ID NO: 1 - protein, 12 aa, Artificial Sequence
  Description of Artificial Sequence Synthetic peptide
YHWYGYTPQNVI

SEQ ID NO: 2 - protein, 5 aa, Artificial Sequence
  Description of Artificial Sequence Synthetic peptide
GGGGC

SEQ ID NO: 3 - protein, 17 aa, Artificial Sequence
  Description of Artificial Sequence Synthetic peptide
YHWYGYTPQNVIGGGGC